SEQUENCE LISTING (44 sequences)

SEQ ID NO: 1 - DNA, 366 bp, Homo sapiens
  CDS (1)..(366): NI-502.4P3 variable heavy chain (VH) sequence
gaggtgcagctggtgcagtctggagcagaggtgaagaagcccggggagtctctgaagatctcctgtaaggtttctggatacgactttagcaagtactggatcggctgggtgcgccagatgcccgggaaaggcctggagtggatggggctcatctatcctggtgactctgataccagatatagtccatctttggaagggcaagtcagcatctcagccgacaagtccatcagcgccgcctacttgcaattcagcagcctgaaggcctcggacagcgccatgtattattgtgtgaggaagggatatagtgcctaccttggagggtggttcgacccctggggccagggaaccctggtcaccgtctcctcg

SEQ ID NO: 2 - protein, 122 aa, Homo sapiens
EVQLVQSGAEVKKPGESLKISCKVSGYDFSKYWIGWVRQMPGKGLEWMGLIYPGDSDTRYSPSLEGQVSISADKSISAAYLQFSSLKASDSAMYYCVRKGYSAYLGGWFDPWGQGTLVTVSS

SEQ ID NO: 3 - protein, 5 aa, Homo sapiens
KYWIG

SEQ ID NO: 4 - protein, 9 aa, Homo sapiens
LIYPGDSDT

SEQ ID NO: 5 - protein, 13 aa, Homo sapiens
KGYSAYLGGWFDP

SEQ ID NO: 6 - DNA, 339 bp, Homo sapiens
  CDS (1)..(339): NI-502.4P3 variable light chain (VL) sequence
gacatccagttgacccagtctccagactccctggctgcgtctctgggcgagagggtcaccatcaactgcaggtccagccagaatattttttacaactccaacaataagaacttcttagcttggtatcagcagaaagcaggacagtctcctaagttgcttatttactgggcttctacgcgggaatccggggtccctgaccgattcagtggcagcggctctgggacagatttcactctcaccatcagcggcctgcaggctggagatgtggcagtttatttctgtcagcaatattttactgatgctcccactttcggcggagggaccaaggtggagatcaaa

SEQ ID NO: 7 - protein, 113 aa, Homo sapiens
DIQLTQSPDSLAASLGERVTINCRSSQNIFYNSNNKNFLAWYQQKAGQSPKLLIYWASTRESGVPDRFSGSGSGTDFTLTISGLQAGDVAVYFCQQYFTDAPTFGGGTKVEIK

SEQ ID NO: 8 - protein, 17 aa, Homo sapiens
RSSQNIFYNSNNKNFLA

SEQ ID NO: 9 - protein, 7 aa, Homo sapiens
WASTRES

SEQ ID NO: 10 - protein, 9 aa, Homo sapiens
QQYFTDAPT

SEQ ID NO: 11 - DNA, 378 bp, Homo sapiens
  CDS (1)..(378): NI-502.31B6 variable heavy chain (VH) sequence
gaggtgcagctgttgcagtctgggggaggcgtggtccagcctggggggtccctgagactctcctgtgaagcctctggattcaccttcagttcctattctatgcactgggtccgccaggctccaggcaaggggctggaatggctgacacttatctcatataatggagtcaataagtattacgcaaagtccgtgaagggccgattcgccatctccagagacgattccaagagcacactgtttttgcaaatgaacagcctgacaactgacgacacggctgtatattactgtgcgagatgtccaatcccgcgggcggcgtcgtatagaaatggagataattatttcgaattcggccagggcaccctggtcaccgtctcctcg

SEQ ID NO: 12 - protein, 126 aa, Homo sapiens
EVQLLQSGGGVVQPGGSLRLSCEASGFTFSSYSMHWVRQAPGKGLEWLTLISYNGVNKYYAKSVKGRFAISRDDSKSTLFLQMNSLTTDDTAVYYCARCPIPRAASYRNGDNYFEFGQGTLVTVSS

SEQ ID NO: 13 - protein, 5 aa, Homo sapiens
SYSMH

SEQ ID NO: 14 - protein, 17 aa, Homo sapiens
LISYNGVNKYYAKSVKG

SEQ ID NO: 15 - protein, 17 aa, Homo sapiens
CPIPRAASYRNGDNYFE

SEQ ID NO: 16 - DNA, 330 bp, Homo sapiens
  CDS (1)..(330): NI-502.31B6 variable light chain (VL) sequence
caggctgtggtgactcaggagccatcgctctcagtgtcccctggagggacagtcacactcacttgtgccctgagttctgcctcagtctctacttcttcgtaccccagctggtaccagcagaccccaggccagcctccacgcatgctcatccacaacacagacactcgctcttctggtgtccctgatcgcttctctggctccatccttgggaagaaagctgccctcaccatcacgggggcccaggcagatgatgaatctgattactactgtgtggtatatttggggaatggcttttgggtgttcggcggagggaccaagctgaccgtccta

SEQ ID NO: 17 - protein, 110 aa, Homo sapiens
QAVVTQEPSLSVSPGGTVTLTCALSSASVSTSSYPSWYQQTPGQPPRMLIHNTDTRSSGVPDRFSGSILGKKAALTITGAQADDESDYYCVVYLGNGFWVFGGGTKLTVL

SEQ ID NO: 18 - protein, 14 aa, Homo sapiens
ALSSASVSTSSYPS

SEQ ID NO: 19 - protein, 7 aa, Homo sapiens
NTDTRSS

SEQ ID NO: 20 - protein, 10 aa, Homo sapiens
VVYLGNGFWV

SEQ ID NO: 21 - DNA, 348 bp, Homo sapiens
  CDS (1)..(348): NI-502.8H1 variable heavy chain (VH) sequence
gaggtgcagctggtggagtctgggggaggcttggtgcagccgggggggtccctaagactctcctgtgcagcctccggattcacctttagcatctataccatgagctgggtccgccaggctccagggaaggggctggagtgggtctcagctattctcagtgatggtagcacatactatgcagactccgtagagggccgattcaccatctccagagacaattccaagaccaccctgtatctacaaatgaacagcctgacagccgaggacacggccgtatattattgtacaaaacgtgttgaggggagctttcgggggtggggccagggaaccctggtcaccgtctcctcg

SEQ ID NO: 22 - protein, 116 aa, Homo sapiens
EVQLVESGGGLVQPGGSLRLSCAASGFTFSIYTMSWVRQAPGKGLEWVSAILSDGSTYYADSVEGRFTISRDNSKTTLYLQMNSLTAEDTAVYYCTKRVEGSFRGWGQGTLVTVSS

SEQ ID NO: 23 - protein, 5 aa, Homo sapiens
IYTMS

SEQ ID NO: 24 - protein, 16 aa, Homo sapiens
AILSDGSTYYADSVEG

SEQ ID NO: 25 - protein, 8 aa, Homo sapiens
RVEGSFRG

SEQ ID NO: 26 - DNA, 327 bp, Homo sapiens
  CDS (1)..(327): NI-502.8H1 variable light chain (VL) sequence
gaaattgtgatgacacagtctccaggcaccctgtctttgtctccaggggaaagcgccaccctctcctgcagggccagtcagagtgttagcactacctacttagcctggtaccagcagaaacctggccaggctcccaggctcctcatctatggtgcatccagcagggccactggcatcccagacagattcagtggcagtgggtctgggacagacttcactctcacaatcagcagactggagcctgaagattttgcagtgtattactgtcagcagtatggtgggtcacgcttgatcaccttcggccaagggacacgactggagattaaa

SEQ ID NO: 27 - protein, 109 aa, Homo sapiens
EIVMTQSPGTLSLSPGESATLSCRASQSVSTTYLAWYQQKPGQAPRLLIYGASSRATGIPDRFSGSGSGTDFTLTISRLEPEDFAVYYCQQYGGSRLITFGQGTRLEIK

SEQ ID NO: 28 - protein, 12 aa, Homo sapiens
RASQSVSTTYLA

SEQ ID NO: 29 - protein, 7 aa, Homo sapiens
GASSRAT

SEQ ID NO: 30 - protein, 10 aa, Homo sapiens
QQYGGSRLIT

SEQ ID NO: 31 - protein, 11 aa, Artificial Sequence
  epitope recognized by NI-502.4P3
TPPTREPKKVA

SEQ ID NO: 32 - protein, 7 aa, Artificial Sequence
  epitope recognized by NI-502.4P3
PMPDLKN

SEQ ID NO: 33 - protein, 18 aa, Artificial Sequence
  epitope of phosphorylated Tau peptide pS202/pT205 recognized by NI-502.31B6
  MOD_RES (8)..(8): PHOSPHORYLATION
  MOD_RES (11)..(11): PHOSPHORYLATION
SGYSSPGSPGTPGSRSRT

SEQ ID NO: 34 - protein, 18 aa, Artificial Sequence
  epitope of phosphorylated Tau peptide pT212/pS214 recognized by NI-502.8H1
  MOD_RES (9)..(9): PHOSPHORYLATION
  MOD_RES (11)..(11): PHOSPHORYLATION
GTPGSRSRTPSLPTPPTR

SEQ ID NO: 35 - protein, 23 aa, Artificial Sequence
  synthetic phosphorylated Tau peptide
  MOD_RES (8)..(8): PHOSPHORYLATION
  MOD_RES (16)..(16): PHOSPHORYLATION
GAEIVYKSPVVSGDTSPRHLSNV

SEQ ID NO: 36 - protein, 18 aa, Artificial Sequence
  synthetic phosphorylated tau peptide
  MOD_RES (11)..(11): PHOSPHORYLATION
REPKKVAVVRTPPKSPSS

SEQ ID NO: 37 - protein, 18 aa, Artificial Sequence
  synthetic phosphorylated tau peptide
  MOD_RES (11)..(11): PHOSPHORYLATION
SSTGSIDMVDSPQLATLA

SEQ ID NO: 38 - protein, 15 aa, Artificial Sequence
  sequence of peptide 54
PSLPTPPTREPKKVA

SEQ ID NO: 39 - protein, 15 aa, Artificial Sequence
  sequence of peptide 55
TPPTREPKKVAVVRT

SEQ ID NO: 40 - protein, 19 aa, Artificial Sequence
  consensus sequence of peptides 54 and 55
PSLPTPPTREPKKVAVVRT

SEQ ID NO: 41 - protein, 15 aa, Artificial Sequence
  sequence of peptide 61
SRLQTAPVPMPDLKN

SEQ ID NO: 42 - protein, 15 aa, Artificial Sequence
  sequence of peptide 62
TAPVPMPDLKNVKSK

SEQ ID NO: 43 - protein, 15 aa, Artificial Sequence
  sequence of peptide 63
PMPDLKNVKSKIGST

SEQ ID NO: 44 - protein, 23 aa, Artificial Sequence
  consensus sequence of peptides 61, 62 and 63
SRLQTAPVPMPDLKNVKSKIGST

(19) United States
(12) Patent Application Publication
(10) Pub. No.: US 2024/0101654 A1
(43) Pub. Date: Mar. 28, 2024

(54) HUMAN ANTI-TAU ANTIBODIES

(71) Applicant: Neurimmune AG, Schlieren (CH)

(72) Inventors: Fabio MONTRASIO, Herznach (CH); Jan GRIMM, Dübendorf (CH)

(21) Appl. No.: 18/269,996

(22) PCT Filed: Dec. 29, 2021
(86) PCT No.: PCT/EP2021/087811

§ 371 Date: Jun. 28, 2023

(30) Foreign Application Priority Data
Dec. 29, 2020 (EP) 20217601.2

(51) Int. Cl. C07K 16/18; A61P 25/28; G01N 33/577

(52) U.S. Cl. CPC C07K 16/18; A61P 25/28; G01N 33/577; C07K 2317/21; C07K 2317/92; G01N 2800/28

(57) ABSTRACT

Provided are novel human-derived monoclonal Tau specific antibodies as well as fragments, derivatives and variants thereof as well as methods related thereto. Polynucleotides, vectors, host cells and kits related to the Tau specific antibodies are also provided. The antibodies, immunoglobulin chain(s), as well as binding fragments, derivatives and variants thereof can be used in pharmaceutical and diagnostic compositions for Tau targeted immunotherapy and diagnosis, respectively.

### FIELD OF THE INVENTION

The present invention generally relates to novel human-derived antibodies as well as fragments, derivatives and biotechnological variants thereof that recognize the Tau protein, including pathologically hyperphosphorylated and mutant forms of Tau.

### BACKGROUND OF THE INVENTION

Protein accumulation, modifications and aggregation are pathological aspects of numerous neurodegenerative diseases. Pathologically modified and aggregated Tau including hyperphosphorylated Tau conformers are an invariant hallmark of tauopathies and correlate with disease severity.

Tau is a microtubule-associated protein expressed in the central nervous system with a primary function to stabilize microtubules. There are six major isoforms of Tau expressed mainly in the adult human brain, which are derived from a single gene by alternative splicing. Under pathological conditions, the Tau protein becomes hyperphosphorylated, resulting in a loss of tubulin binding and destabilization of microtubules followed by the aggregation and deposition of Tau in pathogenic neurofibrillary tangles. There is compelling evidence that Tau hyperphosphorylation and the subsequent formation of higher order multimeric structures leads to neuronal dysfunction and death. For example, there is a strong correlation between the extent of tau pathology and the degree of dementia in Alzheimer's disease (AD) patients, and mutations within the tau gene are known to cause forms of frontotemporal lobar degeneration (FTLD) (Khanna et al. Alzheimers Dement. 10 (2016), 1051-1065 and references cited therein). Further disorders related to Tau—collectively referred to as neurodegenerative tauopathies—are for example, Pick's disease (PiD) and corticobasal degeneration (CBD).

Thus, it is important to develop diagnostic tools to identify Tau pathology as well as approaches for preventing and treating human tauopathies.

### SUMMARY OF THE INVENTION

The present invention relates to the embodiments as characterized in the claims, disclosed in the description and illustrated in the Examples and Figures further below. Thus, the present invention relates to Tau-specific human-derived monoclonal antibodies and Tau-binding fragments thereof as well as equivalent synthetic variants and biotechnological derivatives of the antibodies exemplified herein, that recognize the Tau protein, including pathologically hyperphosphorylated forms of Tau.

As illustrated in the Examples, within a complex antibody discovery process fortunately human monoclonal anti-Tau antibodies have been cloned and identified that bind different forms of Tau in brain tissues of patients suffering from tauopathies. In particular, despite initial failures experiments performed within the scope of the present invention were successful in identifying and cloning antibodies that bind pathological hyperphosphorylated Tau filaments in dystrophic neurites, neurofibrillary tangles and neuropil threads in an immunohistochemical (IHC) assay with brain tissue of patients with Alzheimer's Disease (AD), Progressive supranuclear palsy (PSP) and/or Pick's Disease (PiD).

Several anti-Tau antibodies against various epitopes of tau have been cloned and tested in IHC assays, only a few of which were found to bind Tau in brain tissue of patients with AD; see Table I. Fortunately, three antibodies could be identified, i.e. NI-502.4P3, NI-502.31B6, and NI-502.8H1 which bind in all brain tissues, i.e. in brain tissues of patients with AD, with PSP as well as with PiD.

Furthermore, said antibodies have been found to capture Tau and AD-associated Tau in an immunoprecipitation assay with brain extracts of patients with AD. Hence, although several antibodies have been tested in immunoprecipitation assays, only few including antibodies NI-502.4P3, NI-502.31B6, and NI-502.8H1 have been found to bind Tau in brain extracts of patients with AD.

TABLE I
Binding properties of anti-Tau antibodies

| | | IHC | IP | |
|---|---|---|---|---|
| | | Human | Human | IHC |
| Antibody | Epitope | AD | AD | P301L |
| NI-502.A | linear epitope | +/− | − | +/− |
| NI-502.B | conformational epitope | − | − | − |
| NI-502.C | linear epitope | − | − | − |
| NI-502.D | linear epitopes | − | − | +/− |
| NI-502.E | linear epitopes | − | + | +/− |
| NI-502.F | conformational epitope | − | + | − |
| NI-502.4P3 | 217-TPPTREPKKVA-227 (SEQ ID NO: 31) | + | + | + |
| | and 249-PMPDLKN-255 (SEQ ID NO: 32) | | | |
| NI-502.G | phospho-tau | − | + | − |
| NI-502.H | phospho-tau | − | + | − |
| NI-502.I | phospho-tau | − | − | − |
| NI-502.31B6 | pS202/pT205 | + | + | + |
| NI-502.J | phospho-tau | − | − | − |
| NI-502.K | phospho-tau | − | − | + |
| NI-502.L | phospho-tau | − | − | − |
| NI-502.8H1 | pT212/pS214 | + | + | + |
| NI-508.M | phospho-tau | + | + | + |

Furthermore, it is to be noted that conventionally screening methods, e.g. screening for anti-Tau antibodies that bind human Tau in an IHC assay with tissue of transgenic mice hemizygous for Tau P301L (Q-TauP301L) did result in false-positives, i.e. identification of antibodies that did not bind human AD tissue; see for example antibody NI-502.K in Table I.

Thus, the identification of human anti-Tau antibodies that bind human Tau in brain tissues of human patients, and especially in brain tissue of patients having either AD, PSP or PiD is still not routine yet but requires exploring different antigens for screening, biochemical and immunohistochemical assays for antibody identification and validation of binding specificities and importantly access to a large pool of human donors for the high-throughput screening of human immune repertoires that allow the identification of memory B cells expressing such antibodies. Accordingly, novel anti-Tau antibodies have been cloned and identified, which can be used for diagnosing and, potentially, the treatment of at least three different classes of distinct tauopathies.

The binding specificity and EC₅₀ of human-derived, Tau-specific antibodies was determined by indirect ELISA. Antibody NI-502.4P3 (FIG. 2A) specifically recognized the Tau protein with an EC₅₀ of 15.0 nM. Antibody NI-502.31B6 specifically targeted the synthetic phosphorylated peptide Tau pS202/pT205 with an EC₅₀ of 2.0 nM (FIG. 2B) whereas antibody NI-502.8H1 specifically bound the synthetic phosphorylated peptide Tau pT212/pS214 with an EC₅₀ of 2.2 nM (FIG. 2C).

In addition, antibodies NI-502.4P3, NI-502.31B6 and NI-502.8H1 could be shown to deplete seeding-competent tau from AD homogenates; see FIGS. 8A to 8C.

Thus, the experiments performed in accordance with the present invention were successful in identifying anti-Tau antibodies binding Tau under various different conditions and thus are especially useful in laboratory research and/or diagnosis. It is further prudent to expect that due to the mentioned binding specificities, the antibodies and Tau binding fragments thereof of the present invention are useful in therapy approaches, i.e. in the treatment of tauopathies, preferably of AD, PSP and PiD. This is also confirmed by the antibodies' property to reduce tau seeding activity.

In addition, the epitopes of antibody NI-502.4P3 are located adjacent to and in the microtubule binding region (MTBR), respectively, which spans from residues 224-369 of Tau; see, e.g., Horie et al., Brain 144 (2021), 515-527. Antibodies binding an epitope in that upstream region of MTBR demonstrated a significant and selective ability to mitigate tau seeding and a reduction of inducing tau pathology in cellular and in vivo transgenic mice models seeded by human Alzheimer's disease brain extracts; see summary in Horie et al., (2021) and references cited therein.

Accordingly, given its ability to remove seeding-competent tau from AD homogenates, which is more pronounced compared to antibodies NI-502.31B6 and NI-502.8H1, antibody NI-502.4P3 combines several advantageous properties that makes it the preferred antibody of the present invention, including antibodies with equivalent immunological and biological properties such as those having one or two amino acid substitutions in one or more, preferably in no more than one or two of CDRs of antibody NI-502.4P3.

In one embodiment, the antibody of the present invention [NI-502.4P3] may be characterized by the complementarity determining regions (CDRs) or hypervariable regions of the variable heavy (VH) and variable light (VL) chain comprising the amino acid sequence of SEQ ID: 2 and SEQ ID NO: 7 as shown in FIG. 1A and explained in the Figure legend to FIG. 1 below. In another embodiment, the antibody of the present invention [NI-502.31B6] may be characterized by the CDRs or hypervariable regions of the VH and VL chain comprising the amino acid sequence of SEQ ID NO: 12 and SEQ ID NO: 17 as shown in FIG. 1B and explained in the Figure legend to FIG. 1 below. In a further embodiment, the antibody of the present invention [NI-502.8H1] may be characterized by the CDRs or hypervariable regions of the VH and VL chain comprising the amino acid sequence of SEQ ID NO: 22 and SEQ ID NO: 27 as shown in FIG. 1C and explained in the Figure legend to FIG. 1 below.

As shown in Examples 3 and 4, the antibodies of the present invention either recognize an epitope in the Tau protein or an epitope of phosphorylated Tau peptide. Moreover, as shown by IHC and immunoprecipitation assays in Examples 5 and 6, the phosphorylated Tau peptide specific antibodies also recognize pathologically hyperphosphorylated Tau. Thus, in one embodiment, the antibody [NI-502.4P3] binds an epitope comprising the amino acid sequence 217-TPPTREPKKVA-227 (SEQ ID NO: 31) and 249-PMPDLKN-255 (SEQ ID NO: 32). In another embodiment the antibody [NI-502.31B6] recognizes an epitope of phosphorylated Tau peptide pS202/pT205 having the amino acid sequence SGYSSPG(pS)PG(pT)PGSRSRT (SEQ ID NO: 33), wherein the indicated amino acids are phosphorylated. In a further embodiment, the antibody [NI-502.8H1] recognizes an epitope of phosphorylated Tau peptide pT212/pS214 having the amino acid sequence GTPGSRSR(pT)P(pS)LPTPPTR (SEQ ID NO: 34), wherein the indicated amino acids are phosphorylated. Accordingly, the present invention relates to an antibody or binding fragment thereof having the same binding specificity as any one of antibodies NI-502.4P3, NI-502.31B6, and NI-502.8H1, i.e. any antibody which has the immunological characteristics to
(i) bind pathological hyperphosphorylated Tau filaments in dystrophic neurites, neurofibrillary tangles and neuropil threads in an immunohistochemical (IHC) assay with brain tissue of patients with Alzheimer's Disease (AD), Progressive supranuclear palsy (PSP) and/or Pick's Disease (PiD); and
(ii) capture Tau and AD-associated Tau in an immunoprecipitation (IP) assay with brain extracts of patients with AD; and
wherein the antibody or antigen-binding fragment thereof preferably
(iii) recognizes an epitope comprising
   (a) the amino acid sequence 217-TPPTREPKKVA-227 (SEQ ID NO: 31) and 249-PMPDLKN-255 (SEQ ID NO: 32),
   (b) phosphorylated Tau peptide pS202/pT205 having the amino acid sequence SGYSSPG(pS)PG(pT)PGSRSRT (SEQ ID NO: 33), or
   (c) phosphorylated Tau peptide pT212/pS214 having the amino acid sequence GTPGSRSR(pT)P(pS)LPTPPTR (SEQ ID NO: 34).

The mentioned features can be easily determined in accordance with the experiments and assays disclosed in the appended Examples, wherein antibodies NI-502.4P3, NI-502.31B6, and NI-502.8H1, respectively, can be used as reference antibody. Typically, such antibody will compete with the corresponding reference antibody for binding Tau at the same epitope and the peptide, respectively, mentioned above.

Furthermore, sequence analysis, i.e. comparison of the human Tau sequence (NCBI Gene ID: 4137) with the mouse Tau sequence (NCBI Gene ID: 17762) revealed that the binding epitopes of antibody NI-502.4P3 are shared between human and murine Tau proteins, which makes it prudent to assume that antibody NI-502.4P3 also recognizes the murine Tau protein.

While the invention is illustrated and described by way of reference to the human-derived antibody originally obtained in the experiments performed in accordance with the present invention and described in the Examples it is to be understood that the antibody or antibody fragment of the present invention includes synthetic and biotechnological derivatives of an antibody which means any engineered antibody or antibody-like Tau binding molecule, synthesized by chemical or recombinant techniques, which retains one or more of the functional properties of the subject antibody, in particular recognizing and binding Tau, including pathologically hyperphosphorylated forms of Tau in dystrophic neurites, neurofibrillary tangles and neuropil threads in an immunohistochemical (IHC) assay with brain tissue of patients with AD, PSP and/or PiD and capturing Tau and AD-associated Tau in an immunoprecipitation assay with brain extracts of patients with AD. Thus, while the present invention may be described for the sake of conciseness by way of reference to an antibody or antibodies, unless stated otherwise synthetic and biotechnological derivatives thereof as well as equivalent Tau binding molecules are meant and included within the meaning of the term “antibody”.

Further embodiments of the present invention will be apparent from the description and Examples that follow.

### BRIEF DESCRIPTION OF THE DRAWINGS

FIG. 1: Amino acid sequences of the variable regions, i.e. heavy chain and kappa light chain (VH, VL) of anti-Tau specific human antibodies NI-502.4P3 (A), NI-502.31B6 (B), and NI-502.8H1 (C) of the present invention. Framework (FR) and complementarity determining regions (CDRs) are indicated with the CDRs being underlined. The Kabat numbering scheme was used (cf. http://www.bioinf.org.uk/abs/; Kabat et al., U.S. Dept. of Health and Human Services, “Sequence of Proteins of Immunological Interest” (1983) referred to in the mentioned web reference and given in Table II below. Unless otherwise specified, references to the numbering of specific amino acid residue positions in an antibody or Tau-binding fragment, variant, or derivative thereof of the present invention are according to the Kabat numbering system, which however is theoretical and may not equally apply to every antibody of the present invention. For example, depending on the position of the first CDR the following CDRs might be shifted in either direction. Accordingly, in case of any inadvertent errors or inconsistencies regarding indication of CDRs in FIG. 1 and/or the sequence listing the person skilled in the art on the basis of the disclosure content of the present application, i.e. the variable heavy (VH) and variable light (VL) chain amino acid sequences of antibodies NI-502.4P3, NI-502.31B6 and NI-502.8H1 is well in the position to determine the correct CDR sequences in accordance with Kabat, which shall be used for defining the claimed antibody and Tau-binding fragment thereof. Depicted are the variable heavy chain VH and light chain VL sequence of antibody NI-502.4P3 as set forth in SEQ ID NOs: 2 and 7 (A), of antibody NI-502.31B6 as set forth in SEQ ID NOs: 12 and 17 (B), and of antibody NI-502.8H1 as set forth in SEQ ID Nos: 22 and 27. As further explained in the description, within CDRs and/or framework region conservative amino acid substitutions are preferred which take into account the physicochemical properties of the original amino acid either alone or with an adjacent amino acid as illustrated in Mirsky et al., Mol. Biol. Evol. 32 (2014) 806-819 at page 813, FIG. 6 in particular the AB or LG model, for example such that the position of two amino acids is exchanged.

FIG. 2: Binding specificity and EC₅₀ determination for Tau and synthetic phosphorylated Tau peptides. EC₅₀s of human-derived NI-502 antibodies for rTau (▪), Tau pS202/pT205 (▪), Tau pT212/pS214 (←), Tau pT231 (←), Tau pS396/S404 (♦), Tau pS422 (∘) and BSA control (□) were determined by indirect ELISA. Antibody NI-502.4P3 (A) specifically recognized the human Tau protein with binding affinity of 15.0 nM. Antibody NI-502.31B6 (B) specifically targeted the synthetic phosphorylated peptide Tau pS202/pT205 with an EC₅₀ of 2.0 nM whereas antibody NI-502.8H1 specifically bound the synthetic phosphorylated peptide Tau pT212/pS214 with an EC₅₀ of 2.2 nM. For an overview of phosphorylation sites in Tau protein see, e.g., Götz et al., Annu. Rev. Pathol. Mech. Dis. 14 (2019), 239-261.

FIG. 3: Determination of NI-502.4P3 binding epitope by Pepspot™ epitope mapping analysis. To map the epitope within the human Tau protein that is recognized by the human-derived Tau specific antibody NI-502.4P3, pepscan membrane with 108 linear 15-meric peptides with 11 aa overlap between individual peptides covering the entire human Tau protein sequence was used. Antibody NI-502.4P3 binds specifically to five peptide spots (peptides 54, 55, 61, 62 and 63; FIG. 3A). The sequence covered by peptides 54 and 55 is equivalent to aa 213-231 on the human Tau protein, with a shared core sequence of aa 217-227 whereas the sequence covered by peptides 61, 62 and 63 is equivalent to aa 241-263 on the human Tau protein, with a shared core sequence of aa 249-255. Graphical overview of peptides sequences and antibody binding scores to the peptides 54, 55, 61, 62 and 63 for antibody NI-502.4P3 are depicted (FIG. 3B). Overlapping amino acids between peptides being recognized by this antibody are highlighted in gray. Consensus sequences of the Tau protein in which the putative binding epitopes of NI-502.4P3 antibody are localized (highlighted in gray, bottom).

FIG. 4: Binding specificity of antibodies NI-502.4P3, NI-502.31B6 and NI-502.8H1 for hyperphosphorylated Tau filaments in AD brain tissues by immunohistochemical analysis. Neurofibrillary tangles are one of the two neuropathological hallmarks of AD, which are composed mainly of hyperphosphorylated Tau filaments. Hyperphosphorylated Tau filaments are also the major component of dystrophic neurites and neuropil threads, both of which are common neuropathological features in AD. Binding of antibodies NI-502.4P3, NI-502.31B6 and NI-502.8H1 to Tau was characterized by immunohistochemical staining of brain sections from patients with neuropathologically confirmed AD. NI-502.4P3, NI-502.31B6 and NI-502.8H1 antibodies showed staining of dystrophic neurites, neurofibrillary tangles and neuropil threads in human AD brain tissues, whereas antibody staining was absent in human non-neurological control brain tissues. Secondary antibody alone results in no staining in paraffin sections of the tested human tissues (data not shown). Antibodies NI-502.4P3, NI-502.31B6 and NI-502.8H1 were used at 25 nM. Representative images are shown.

FIG. 5: Binding specificity of antibodies NI-502.4P3, NI-502.31B6 and NI-502.8H1 for hyperphosphorylated Tau filaments in AD brain tissues by immunohistochemical analysis. Representative high magnification images of dystrophic neurites, neurofibrillary tangles and neuropil threads in human brain tissues of selected AD cases detected by antibodies NI-502.4P3, NI-502.31B6 and NI-502.8H1. Representative images are shown.

FIG. 6: Binding specificity of antibodies NI-502.4P3, NI-502.31B6 and NI-502.8H1 for hyperphosphorylated Tau filaments in Progressive supranuclear palsy and Pick's Disease brain tissues by immunohistochemical analysis. Neurofibrillary tangles composed of hyperphosphorylated Tau filaments are neuropathological hallmarks of AD and other Tauopathies, such as Pick's Disease and Progressive supranuclear palsy. Hyperphosphorylated Tau filaments are also the major component of dystrophic neurites and neuropil threads, both of which are common neuropathological features in AD and other Tauopathies. Binding of antibodies NI-502.4P3, NI-502.31B6 and NI-502.8H1 to Tau was characterized by immunohistochemical staining of brain sections from patients with neuropathologically confirmed Progressive supranuclear palsy and Pick's Disease. NI-502.4P3, NI-502.31B6 and NI-502.8H1 antibodies showed staining of dystrophic neurites, neurofibrillary tangles and neuropil threads in human Progressive supranuclear palsy and Pick's Disease brain tissues, whereas antibody staining was absent in human non-neurological control brain tissues (see FIGS. 4 and 5). Secondary antibody alone results in no staining in paraffin sections of the tested human tissues (data not shown). Antibodies NI-502.4P3, NI-502.31B6 and NI-502.8H1 were used at 25 nM. Representative images are shown.

FIG. 7: Determination of target binding in solution for antibodies NI-502.4P3, NI-502.31B6 and NI-502.8H1 by immunoprecipitation assays. Immunoblot analysis of immunoprecipitated samples and crude brain tissue homogenates were performed by using the Tau-specific, commercially available, mouse monoclonal antibody Tau 12. Antibodies NI-502.4P3, NI-502.31B6 and NI-502.8H1 specifically capture both Tau and disease-associated Tau (PFHTau) in human non-neurological control's (ctrl. brain homogenate) and Alzheimer Disease's patient's (AD brain homogenate) brain extracts, respectively. Antibodies NI-502.4P3, NI-502.31B6 and NI-502.8H1 preferentially capture disease-associated Tau in Alzheimer Disease's patient's brain homogenate as compared to endogenous Tau in non-neurological control's brain homogenate. As additional controls for equal Tau distribution, crude brain tissue homogenates were loaded.

FIG. 8: Immunodepletion of seeding competent Tau from AD brain homogenate by anti-tau antibodies NI-502.4P3, NI-502.31B6 and NI-502.8H1. Tau aggregation in HEK293T Tau biosensor cells using AD brain homogenate from a selected donor that had been immunodepleted with increasing concentrations of NI-502.4P3 (A), NI-502.31B6 (B) or NI-502.8H1 (C) antibodies. The derived IC₅₀ values were 6.5, and 7.1 μg/mL, for donors NI-502.4P3 and NI-502.8H1, respectively. Due to the obtained curve fitting, no IC₅₀ value could be determined for NI-502.31B6. Data were fitted to a non-linear regression curve; each antibody concentration was tested in duplicate, error bars represent standard deviation. AD, Alzheimer's disease; FRET, fluorescence resonance energy transfer.

### DETAILED DESCRIPTION OF THE INVENTION

Generally, the present invention relates to human-derived monoclonal anti-tau antibodies that demonstrate the immunological characteristics of any one of the anti-tau antibodies illustrated in the Examples and Figures further below. Due to their unique binding specificities, i.e. binding Tau in brain tissue of patients with Alzheimer's Disease (AD), Progressive supranuclear palsy (PSP) as well as Pick's Disease (PiD) and capturing Tau and AD-associated Tau in an immunoprecipitation assay with brain extracts of patients with AD, the antibodies are especially useful in laboratory research and/or diagnosis of diseases related to Tau, i.e. tauopathies, preferably in the diagnosis of AD, PSP and/or PiD. It is further prudent to expect that due to the mentioned binding specificities, and due to their human origin, i.e. maturation of the original antibodies in the human body, the antibodies and Tau binding fragments thereof of the present invention are useful in therapy of tauopathies, in particular, in the treatment of AD, PSP and/or PiD.

The antibodies and antigen-binding fragments thereof of the present invention bind Tau including pathologically hyperphosphorylated forms of Tau in dystrophic neurites, neurofibrillary tangles and neuropil threads in an immunohistochemical (IHC) assay with the brain tissues mentioned above. Neurofibrillary tangles composed of hyperphosphorylated Tau filaments are neuropathological hallmarks of AD, PSP and PiD. Hyperphosphorylated Tau filaments are also the major components of dystrophic neurites and neuropil threads, both of which are common neuropathological features in AD, PSP and PiD.

Phosphorylation of Tau occurs at about 30 of 79 potential serine (Ser) and threonine (Thr) phosphorylation sites. Tau is highly phosphorylated during brain development. The degree of phosphorylation declines in adulthood. Some of the phosphorylation sites are located within the microtubule binding domains of Tau, and it has been shown that an increase of Tau phosphorylation negatively regulates the binding of microtubules. For example, Ser262 and Ser396, which lie within or adjacent to microtubule binding motifs, are hyperphosphorylated in the Tau proteins of the abnormal paired helical filaments (PHFs), a major component of the neurofibrillary tangles (NFTs) in the brain of AD patients.

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2; Second edition published 2006, ISBN 0-19-852917-1 978-0-19852917-0.

Furthermore, regarding Tau protein and anti-Tau antibodies, their recombinant production in a host cell, purification, modification, formulation in a pharmaceutical composition and therapeutic use as well as terms and feature common in the art can be relied upon by the person skilled in art when carrying out the present invention as claimed; see, e.g., Antibodies A Laboratory Manual 2^{nd} edition, 2014 by Cold Spring Harbor Laboratory Press, Cold Spring Harbor, New York, USA, wherein also antibody purification and storage; engineering antibodies, including use of degenerate oligonucleotides, 5′-RACE, phage display, and mutagenesis, immunoblotting protocols and the latest screening and labeling techniques are described.

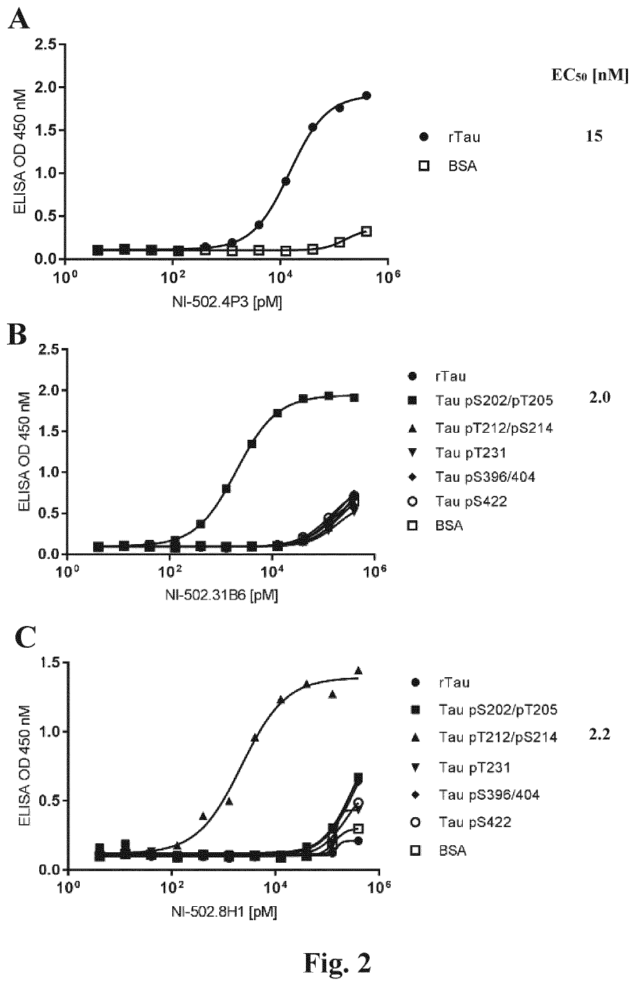

As further illustrated in the Examples and in the Figures, e.g., in FIG. 2, the antibodies of the present invention have been originally isolated from human donors and are shown to specifically recognize human Tau. By “specifically recognizing tau”, “binding tau”, “antibody specific to/for tau” and “anti-tau antibody” is meant specifically, generally, and collectively, antibodies to the native form of tau, or aggregated or pathologically modified tau isoforms. Provided herein are human antibodies selective for full-length, pathologically phosphorylated and aggregated forms. Binding specificity of an anti-tau antibody may be measured, for example, by determination of binding affinity to various tau peptides, as measured by the assay in the Example 3 or 5 herein, or by an equivalent assay.

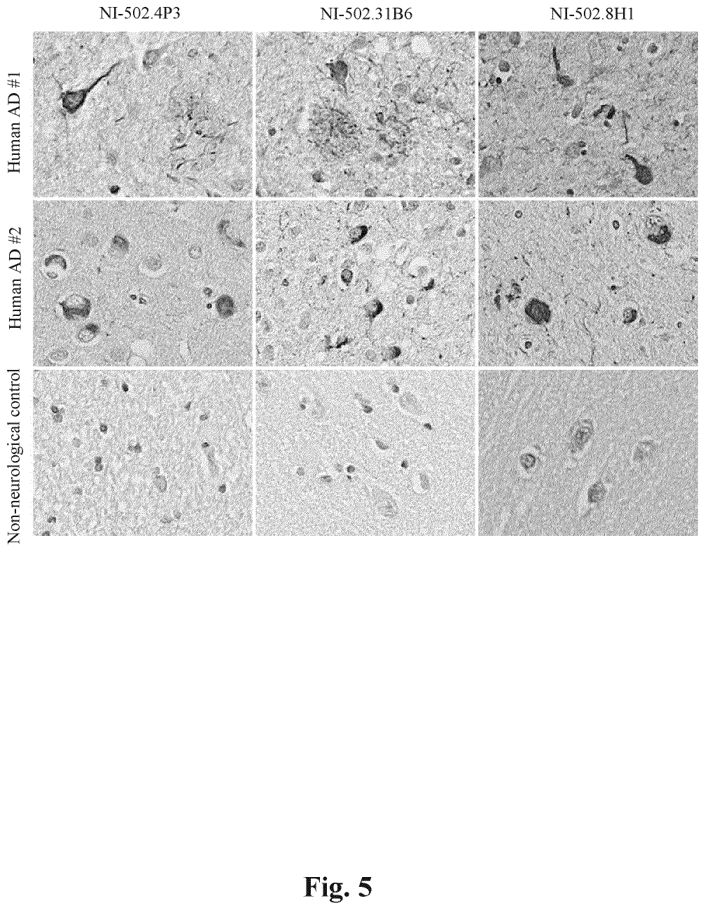

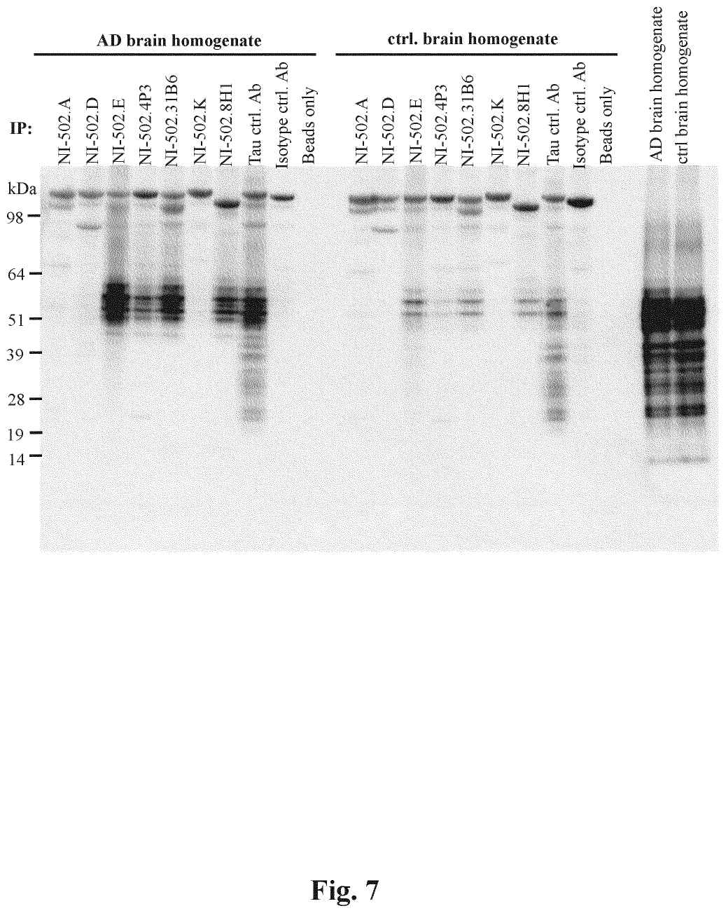

Therefore, in one embodiment the anti-Tau antibody and Tau binding fragment of the present invention recognizes human Tau in various tissues as mentioned above. Binding characteristics such as specificity and affinity of the antibodies of the present invention have been tested in several experimental assays as described and shown herein, e.g., in Examples 3 and 5 and in FIGS. 2 and 4 to 7. In this context, in order to obtain a measure of the binding affinity, the EC₅₀ of the antibodies of the invention in the ELISA performed in Example 3 was determined. As demonstrated, the antibodies of the present invention display a particularly high apparent binding affinity as determined by the EC₅₀ value. The term “EC₅₀”, in the context of an in vitro or in vivo assay using an antibody or antigen-binding fragment thereof, refers to the concentration of an antibody or an antigen-binding fragment thereof that induces a response that is 50% of the maximal response, i.e., halfway between the maximal response and the baseline.

In particular, the EC₅₀ as determined by indirect ELISA of antibody NI-502.4P3 for binding the Tau protein is 15.0 nM (FIG. 2A). In another embodiment, the EC₅₀ of antibody NI-502.31B6 for the synthetic phosphorylated peptide Tau pS202/pT205 is 2.0 nM (FIG. 2B) and of antibody NI-502.8H1 for the synthetic phosphorylated peptide Tau pT212/pS214 is 2.2 nM (FIG. 2C).

Thus, the antibody of the present invention is characterized by having an EC₅₀ of about 15 nM or by having a greater binding affinity, i.e. a lower EC₅₀ value for Tau or by having an EC₅₀ of about 2 nM or by having a greater binding affinity, i.e. a lower EC₅₀ value for phosphorylated Tau, as measured by indirect ELISA or an equivalent assay. However, also depending on the antibody format, for example whether an IgG1, IgG4 or antibody fragments are used, like Fab fragments, the EC₅₀ values may deviate and may be for example higher than the values mentioned above and in the Examples. Accordingly, in this context the term “about” means a value which may differ from the value determined for the reference antibody in the Examples, the difference being preferably less than one order of magnitude and most preferably within the same order of magnitude, for example the EC₅₀ may be the reference value±10 nM.

As mentioned above, under pathological conditions, Tau protein becomes hyperphosphorylated, resulting in a loss of tubulin binding and destabilization of microtubules followed by the aggregation and deposition of Tau in pathogenic neurofibrillary tangles. Those aggregates can further drive misfolding of non-pathological Tau in a prion-like manner. Although templated misfolding occurs intracellularly, some of these Tau species may be released into the extracellular space and internalized by neighboring cells where they can act as a seed for intracellular Tau aggregation. In this self-perpetuating process, seeded aggregation of conformationally altered Tau may spread along neuronal networks to interconnected neurons and adjacent glial cells from one neuroanatomically connected brain region to another and thus propagate Tau pathology; see for example Sopko et al., Neurobiol. Dis. 146 (2020), 105120, doi: 10.1016/j.nbd.2020.105120 and Kfoury et al., J. Biol. Chem. 287 (2012), 19440-19451. The specific form of Tau aggregate which facilitates this cell-to-cell spread of Tau aggregates is referred to as “Tau seeds” and the activity as “seeding activity”, since this form of Tau aggregate seeds or nucleates Tau aggregation in the cell it enters (i.e. the “recipient cell”). A “seed” nucleates aggregation of other proteins with a similar aggregation domain. On the basis of the hypothesis that a seed-competent, extracellular Tau species can transit from cell to cell and propagate Tau pathology throughout the brain, anti-Tau approaches are preferred which intervene with Tau spread.

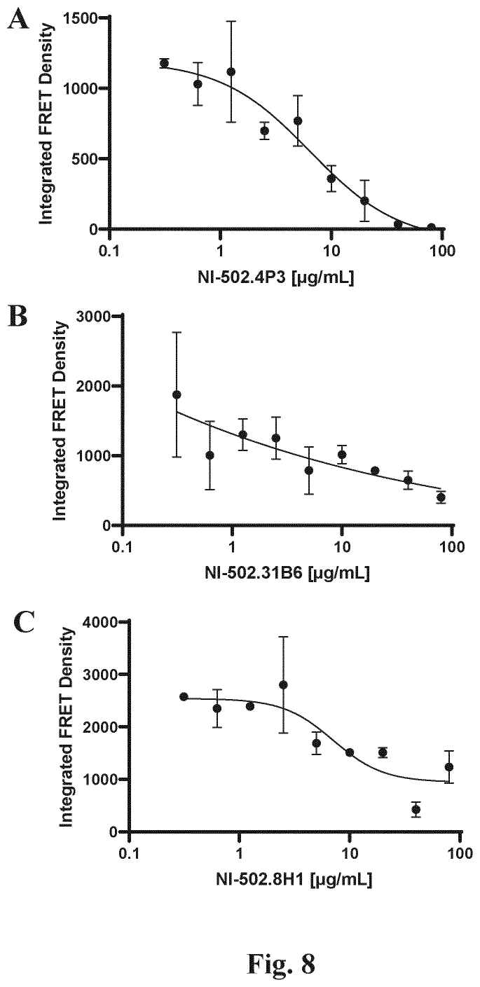

As show by the experiments performed within the scope of the present invention, antibodies NI-502.4P3, NI-502.31B6 and NI-502.8H1 immunodepleted seed-competent Tau from AD brain homogenates; see Example 7 and FIGS. 8A to C. Derived IC₅₀ values from the brain homogenate seeding experiments ranged from 6 to 8 μg/mL, wherein the derived IC₅₀ values were 6.5, and 7.1 μg/mL, for antibodies NI-502.4P3 and NI-502.8H1, respectively. The “IC₅₀” (half maximal inhibitory concentration) value, in the context of an assay using an antibody or an antigen-binding fragment thereof, is a quantitative measure that indicates how much of a particular antibody or antigen-binding fragment thereof is needed to inhibit, in vitro, a given biological process or biological component by 50%.

Thus, the antibody or antigen-binding fragment thereof of the present invention is able to block Tau seeding activity in a cellular Tau aggregation assay and thus can be expected to inhibit or mitigate Tau cell-to-cell spreading. In one embodiment, the IC₅₀ value ranges from about 5 to 10 μg/ml, preferably from about 6 to 8 μg/mL, and is in particular 6.5 μg/mL [NI-502-4P3] or 7.1 μg/mL [NI-502-8H1].

Thus, in a preferred embodiment, the antibody or antigen-binding fragment thereof of the present invention disclosed herein is capable of
(i) binding pathological hyperphosphorylated Tau filaments in dystrophic neurites, neurofibrillary tangles and neuropil threads in an immunohistochemical (IHC) assay with brain tissue of patients with Alzheimer's Disease (AD), Progressive supranuclear palsy (PSP) and/or Pick's Disease (PiD);
(ii) capturing Tau and AD-associated Tau in an immunoprecipitation (IP) assay with brain extracts of patients with AD;
(iii) blocking Tau seeding activity in a cellular Tau aggregation assay; and optionally
(iv) recognizing mouse Tau protein.

Cellular tau aggregation assays for determining blocking Tau seeding activity are known in the art; see, e.g., Sopko et al. (2020) and Kfoury et al. (2012), supra, and international applications WO 2014/008404 A1 and WO 2014/089104 A1. Preferably, the method as described in Example 7 is used, i.e. a cellular Tau aggregation assay which uses HEK293T biosensor cells which stably express the repeat domains (RD) of tau protein with a P301S mutation fused to either CFP or YFP, wherein the sample to be analyzed is a Alzheimer's Disease brain homogenate.

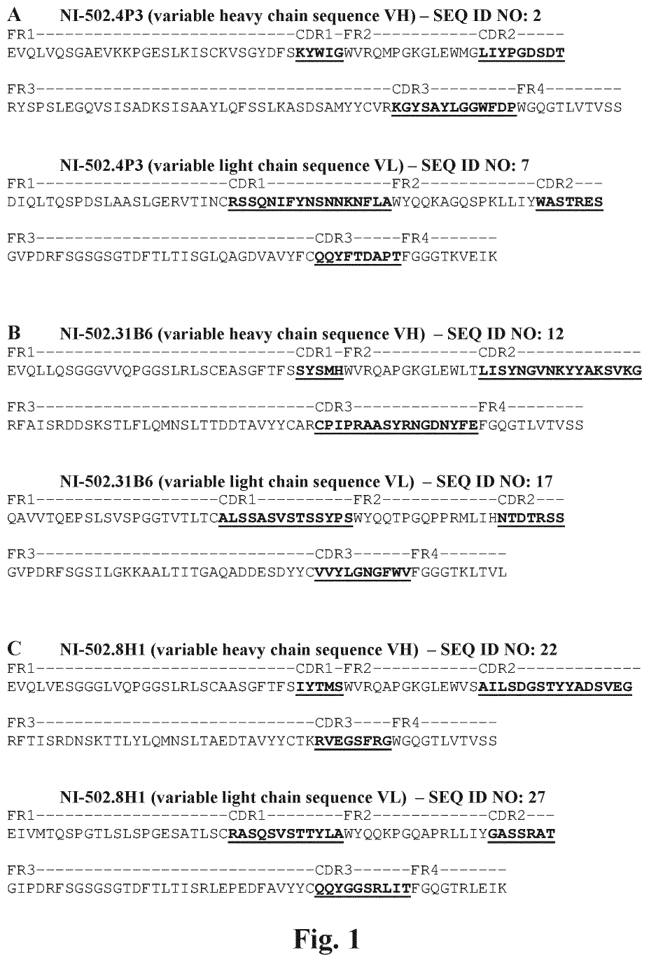

The present invention is illustrated with anti-Tau antibodies and antigen-binding fragments thereof which are characterized by comprising in their variable region, i.e. binding domain, the variable heavy (V_{H}) and variable light (V_{L}) chain having the amino acid sequences depicted in FIGS. 1A, B and C, respectively. The corresponding nucleotide and amino acid sequences are set forth in Table II below.

As always, the variable domains of each chain contain three hypervariable loops named complementarity determining regions (CDRs, CDR-1, -2, and -3). The CDRs are separated by structurally conserved regions called framework regions (FR-1, -2, -3, and -4) that form a “core” ß-sheet structure displaying these loops on the surface of the variable domain. The length and composition of the CDR sequences are highly variable, especially in the CDR3. The CDRs are approximated to the paratope of the antibody that interacts with the antigen and therefore contains the antigen-binding residues. Accordingly, it is common to define an antibody by its six CDRs. Exemplary sets of CDRs in the above amino acid sequences of the V_{H} and V_{L} chains are depicted in FIGS. 1A, B and C. However, as discussed in the following the person skilled in the art is well aware of the fact that in addition or alternatively CDRs may be used, which differ in their amino acid sequence from those set forth in any one of FIGS. 1A, B and C by one, two, three or even more amino acids, especially in case of CDR2 and CDR3. As mentioned in the Figure legend of FIG. 1, the person skilled in the art can easily identify the CDRs according to common principles, for example as summarized in www.bioinf.org.uk/abs. In this context, while the CDRs of the antibodies depicted in FIG. 1 are indicated according to Kabat et al. the person skilled in the art knows that a number of definitions of the CDRs are commonly in use, i.e. the
(i) Kabat definition based on sequence variability, which is the most commonly used;
(ii) Chothia definition based on the location of the structural loop regions;
(iii) AbM definition as a compromise between the two used by Oxford Molecular's AbM antibody modelling software; and
(iv) Contact definition that has been recently introduced by and is based on an analysis of the available complex crystal structures. This definition is likely to be the most useful for performing mutagenesis to modify the affinity of an antibody since these are residues which take part in interactions with the antigen. For lists of CDR residues making contact in each antibody with summary data for each CDR see, e.g., www.bioinf.org.uk/abs which also refers to antibody modelling software such as abYmod available at abymod.abysis.org.

Table II below depicts the relation between the CDR positions defined by the different concepts.

TABLE II
Different concepts of CDR definitions.

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L51 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹some of these definitions (particularly for Chothia loops) vary depending on the individual publication examined;
²any of the numbering schemes can be used for these CDR definitions, except the contact definition uses the Chothia or Martin (Enhanced Chothia) definition;
³the end of the Chothia CDR-H1 loop when numbered using the Kabat numbering convention varies between H32 and H34 depending on the length of the loop. (This is because the Kabat numbering scheme places the insertions at H35A and H35B.)

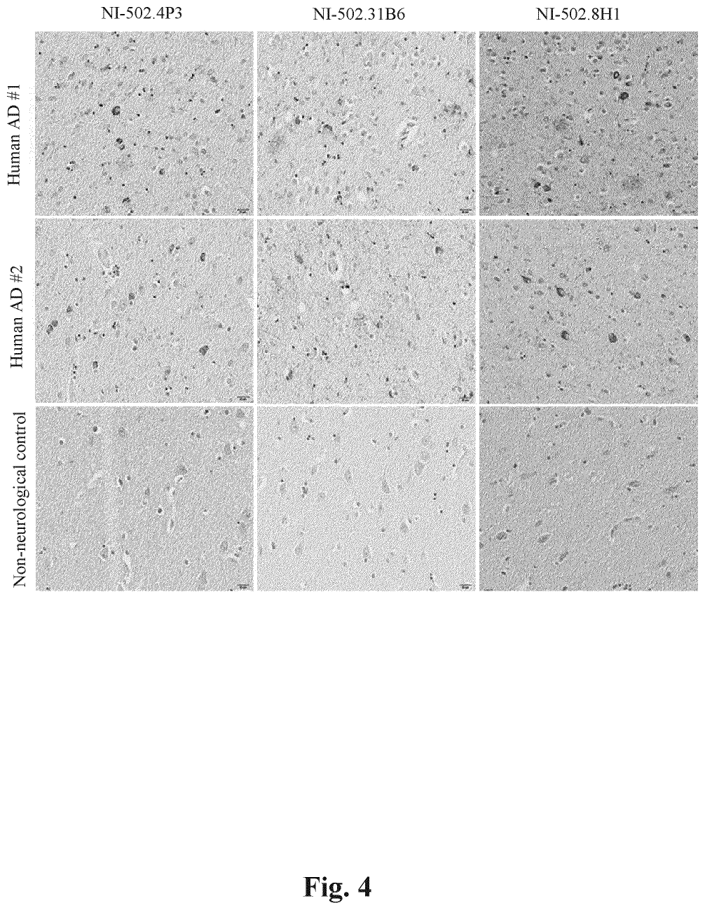

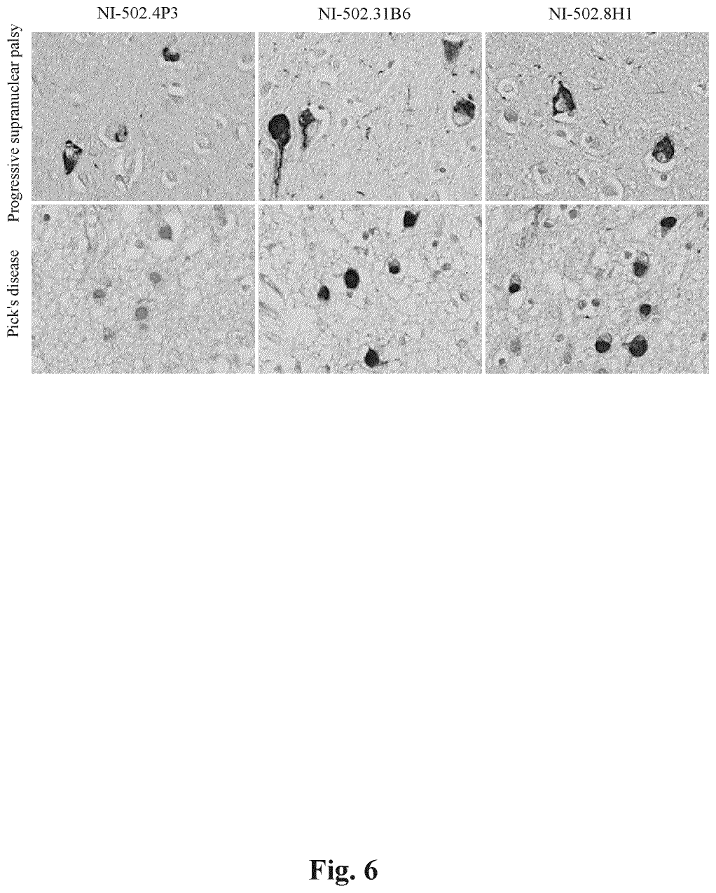

For the mentioned definitions see also Kontermann and Dübel (eds.), Antibody Engineering Vol. 2, DOI 10.1007/978-3-642-01147-4_3, #Springer-Verlag Berlin Heidelberg 2010, in particular Chapter 3, Protein Sequence and Structure Analysis of Antibody Variable Domains at pages 33-51 and Dondelinger et al., Front. Immunol. 9 (2018), 2278 specifically dealing with understanding the significance and implications of antibody numbering and antigen-binding surface/residue definition; see, e.g., Dondelinger et al., FIG. 4 and FIG. 6 illustrating the disparity in the classical CDR definitions according to Kabat supra, Chothia (Chothia and Lesk, J. Mol. Biol. 196 (1987), 901-917), Contact (MacCallum et al, J. Mol. Biol. 262 (1996), 732-745) and IMGT (IMGT®, the international ImMunoGeneTics information System®, www.imgt.org). The AbM definition is a compromise between the two used by Oxford Molecular's AbM antibody modelling software.

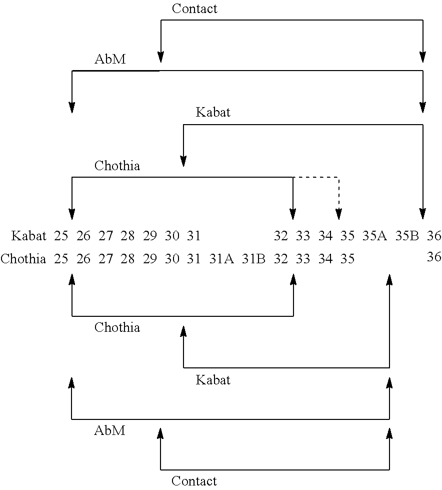

This above diagram illustrates the alternative definitions for CDR-H1 (VH-CDR1). The Kabat and Chothia numbering schemes are shown horizontally and the Kabat, Chothia, AbM and Contact definitions of the CDRs are shown with arrows above and below the two numbering schemes.

In one embodiment, the present invention relates to a human-derived recombinant monoclonal anti-Tau antibody or Tau binding fragment, synthetic derivative, or biotechnological derivative of antibody NI-502.4P3, NI-502.31B6 or NI-502.8H1, wherein the antibody, fragment or derivative thereof comprises a variable heavy (VH) chain comprising VH complementarity determining regions (CDRs) 1, 2, and 3, and a variable light (VL) chain comprising VL CDRs 1, 2, and 3 as defined by Kabat, wherein NI-502.4P3
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions; or
NI-502.31B6
(g) VH-CDR1 comprises the amino acid sequence of SEQ ID NO: 13 or a variant thereof, wherein the variant comprises one or two amino acid substitutions,
(h) VH-CDR2 comprises the amino acid sequence of SEQ ID NO: 14 or a variant thereof, wherein the variant comprises one or two amino acid substitutions,
(i) VH-CDR3 comprises the amino acid sequence of SEQ ID NO: 15 or a variant thereof, wherein the variant comprises one or two amino acid substitutions,
(j) VL-CDR1 comprises the amino acid sequence of SEQ ID NO: 18 or a variant thereof, wherein the variant comprises one or two amino acid substitutions,
(k) VL-CDR2 comprises the amino acid sequence of SEQ ID NO: 19 or a variant thereof, wherein the variant comprises one or two amino acid substitutions, and
(l) VL-CDR3 comprises the amino acid sequence of SEQ ID NO: 20 or a variant thereof, wherein the variant comprises one or two amino acid substitutions; or
NI-502.8H1
(m) VH-CDR1 comprises the amino acid sequence of SEQ ID NO: 23 or a variant thereof, wherein the variant comprises one or two amino acid substitutions,
(n) VH-CDR2 comprises the amino acid sequence of SEQ ID NO: 24 or a variant thereof, wherein the variant comprises one or two amino acid substitutions,
(o) VH-CDR3 comprises the amino acid sequence of SEQ ID NO: 25 or a variant thereof, wherein the variant comprises one or two amino acid substitutions,
(p) VL-CDR1 comprises the amino acid sequence of SEQ ID NO: 28 or a variant thereof, wherein the variant comprises one or two amino acid substitutions,
(q) VL-CDR2 comprises the amino acid sequence of SEQ ID NO: 29 or a variant thereof, wherein the variant comprises one or two amino acid substitutions, and
(r) VL-CDR3 comprises the amino acid sequence of SEQ ID NO: 30 or a variant thereof, wherein the variant comprises one or two amino acid substitutions.

The antibody or binding fragment thereof of the present invention can be further characterized by binding pathological hyperphosphorylated Tau filaments in dystrophic neurites, neurofibrillary tangles and neuropil threads in an IHC assay with brain tissue of patients with AD, PSP and/or PiD as well as by capturing Tau and AD-associated Tau in an IP assay with brain extracts of patients with AD.

In a further embodiment, the antibody or binding fragment thereof shows the above mentioned binding specificities, recognizes an epitope comprising the amino acid sequence 217-TPPTREPKKVA-227 (SEQ ID NO: 31) and 249-PMPDLKN-255 (SEQ ID NO: 32) and comprises a variable heavy (VH) chain comprising VH complementarity determining regions (CDRs) 1, 2, and 3, and a variable light (VL) chain comprising VL CDRs 1, 2, and 3, wherein
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions;
   recognizes an epitope of phosphorylated Tau peptide pS202/pT205 having the amino acid sequence SGYSSPG(pS)PG(pT)PGSRSRT (SEQ ID NO: 33) and comprises a VH chain comprising VH CDRs 1, 2, and 3, and a VL chain comprising VL CDRs 1, 2, and 3, wherein
(g) VH-CDR1 comprises the amino acid sequence of SEQ ID NO: 13 or a variant thereof, wherein the variant comprises one or two amino acid substitutions,
(h) VH-CDR2 comprises the amino acid sequence of SEQ ID NO: 14 or a variant thereof, wherein the variant comprises one or two amino acid substitutions,
(i) VH-CDR3 comprises the amino acid sequence of SEQ ID NO: 15 or a variant thereof, wherein the variant comprises one or two amino acid substitutions,
(j) VL-CDR1 comprises the amino acid sequence of SEQ ID NO: 18 or a variant thereof, wherein the variant comprises one or two amino acid substitutions,
(k) VL-CDR2 comprises the amino acid sequence of SEQ ID NO: 19 or a variant thereof, wherein the variant comprises one or two amino acid substitutions, and
(l) VL-CDR3 comprises the amino acid sequence of SEQ ID NO: 20 or a variant thereof, wherein the variant comprises one or two amino acid substitutions; or
   recognizes an epitope of phosphorylated Tau peptide pT212/pS214 having the amino acid sequence GTPGSRSR(pT)P(pS)LPTPPTR (SEQ ID NO: 34) and comprises a VH chain comprising VH CDRs 1, 2, and 3, and a VL chain comprising VL CDRs 1, 2, and 3, wherein
(m) VH-CDR1 comprises the amino acid sequence of SEQ ID NO: 23 or a variant thereof, wherein the variant comprises one or two amino acid substitutions,
(n) VH-CDR2 comprises the amino acid sequence of SEQ ID NO: 24 or a variant thereof, wherein the variant comprises one or two amino acid substitutions,
(o) VH-CDR3 comprises the amino acid sequence of SEQ ID NO: 25 or a variant thereof, wherein the variant comprises one or two amino acid substitutions,
(p) VL-CDR1 comprises the amino acid sequence of SEQ ID NO: 28 or a variant thereof, wherein the variant comprises one or two amino acid substitutions,
(q) VL-CDR2 comprises the amino acid sequence of SEQ ID NO: 29 or a variant thereof, wherein the variant comprises one or two amino acid substitutions, and
(r) VL-CDR3 comprises the amino acid sequence of SEQ ID NO: 30 or a variant thereof, wherein the variant comprises one or two amino acid substitutions.

As mentioned above, sequence analysis, i.e. comparison of the human Tau sequence (Gene ID: 4137) with the mouse Tau sequence (Gene ID: 17762) revealed that the binding epitopes of antibody NI-502.4P3 are shared between human and murine Tau proteins, which makes it prudent to assume that this antibody also recognizes the murine Tau protein. Accordingly, in one embodiment, the antibody or antigen-binding fragment thereof of the present invention recognizes and is thus capable of binding murine Tau.

In addition, or alternatively the antibody or antigen-binding fragment thereof of the present invention can be characterized in that:
(i) the VH chain comprises the amino acid sequence depicted in SEQ ID NO: 2 or a variant thereof, wherein the variant comprises one or more amino acid substitutions; and
(ii) the VL comprises the amino acid sequence depicted in SEQ ID NO: 7, or a variant thereof, wherein the variant comprises one or more amino acid substitutions; or
(iii) the VH comprises the amino acid sequence depicted in SEQ ID NO: 12 or a variant thereof, wherein the variant comprises one or more amino acid substitutions; and
(iv) the VL comprises the amino acid sequence depicted in SEQ ID NO: 17, or a variant thereof, wherein the variant comprises one or more amino acid substitutions; or
(v) the VH comprises the amino acid sequence depicted in SEQ ID NO: 22 or a variant thereof, wherein the variant comprises one or more amino acid substitutions; and
(vi) the VL comprises the amino acid sequence depicted in SEQ ID NO: 27, or a variant thereof, wherein the variant comprises one or more amino acid substitutions.

In a preferred embodiment, the VH and VL chain amino acid sequences are at least 90% identical to SEQ ID NO: 2 and 7, respectively, to SEQ ID NO: 12 and 17, respectively or SEQ ID NO: 22 and 27, respectively.

In these embodiments, preferably one or more of the CDRs according to the Kabat definition are maintained substantially unchanged. However, under the simplified assumption that the paratope corresponds to the CDRs, the Chothia definition of the CDRs may be used in addition or alternatively as they correlate very well with the structural loops present in the variable regions. Thus, in order to provide anti-Tau antibodies equivalent to subject antibodies NI-502.4P3, NI-502.31B6, and NI-502.8H1, preferably at least one or two of said one or more, preferably not more than two amino acid substitutions if made in the CDRs as defined according to Kabat are made outside the CDRs as defined by Chothia and/or IMGT and most preferably outside the overlap of the CDRs as defined according to Kabat and Chothia.

For example, regarding amino acid substitutions within the CDRs, variable heavy and light chain and framework amino acid sequences, respectively, preferably conservative amino acid substitutions are performed for example in accordance with the most frequently exchanged amino acids as analyzed and described by Mirsky et al., Mol. Biol. Evol. 32 (2014), 806-819; see FIG. 6 at page 813 of Mirsky et al. In particular, within VH-CDR1, S may be substituted with T; within VH-CDR3, V may be substituted with E, T may be substituted with S and/or M may be substituted with V; within VL-CDR1, R may be substituted with K, R may be substituted with E, and/or T may be substituted; within VL-CDR2, S may be substituted with A and/or A may be substituted with G; and in VL-CDR3, P may be substituted with S. As mentioned, preferably amino acid substitutions are selected which belong to the same category in either or preferably both models LG and AB shown in FIG. 6 of Mirsky et al. (2014), supra, with the LG model being preferred for the tendency to keep amino acid properties, and wherein the amino acid substitutions are selected preferably such that the physiochemical properties of the original amino acid is substantially maintained, i.e. hydrophobic, polar or charged property or for example that in case two or more amino acid substitutions are performed, they compensate each other so as to provide the physicochemical property of the surface all together. In a preferred embodiment, the antibody of the invention comprises a variant of the amino acid sequence of the VH and/or VL region which is at least 90%, 95%, 96%, 97%, 98%, 99% or 100% identical to the VH and VL regions depicted in FIGS. 1A, B and C, respectively.

Of course, besides theoretical considerations also experimental approaches exist for identifying CDR variants within a reasonable time and undue burden. For example, Tiller et al., in Front Immunol. 8 (2017), 986 describe facile affinity maturation of antibody variable domains using natural diversity mutagenesis. Indeed, already a few years earlier Rajpal et al., in PNAS 102 (2005), 8466-8471 reported a general method for greatly improving the affinity of antibodies by using combinatorial libraries and illustrated their method with anti-TNF-α antibody D2E7 (HUMIRA©) identifying 38 substitutions in 21 CDR positions that resulted in higher affinity binding to TNF-α. More recently, Cannon et al., in PLOS Computational Biology, https://doi org/10.1371/journal.pcbi.1006980 May 1, 2019 described experimentally guided computational antibody affinity maturation with de novo docking, modelling and rational design in silico affinity maturation, together with alanine scanning, that allowed fine-tuning the protein-protein docking model to subsequently enable the identification of two single-point mutations that increase the affinity of a hybridoma-derived antibody, AB1 for its antigen murine CCL20.

Accordingly, though each antibody is unique and may have distinct features, nevertheless once a lead candidate has been provided the person skilled in the art in consideration of the teaching of the present invention as disclosed in the present application, as well as in view of the computational design and experimental approaches developed so far is able to arrive at equivalent anti-Tau antibodies which keep the desired features of the antibody such as those described for the anti-Tau antibodies illustrated in the Examples and specifically defined in the claims. In this context, it is well understood that the variant antibody substantially maintains the binding specificity of the parent antibody, for example recognizing and binding Tau, including pathologically hyperphosphorylated forms of Tau in dystrophic neurites, neurofibrillary tangles and neuropil threads in an IHC assay with brain tissue of patients with AD, PSP and/or PiD and capturing Tau and AD-associated Tau in an immunoprecipitation assay with brain extracts of patients with AD or for example competing with the parent antibody, i.e. with any one of antibodies NI-502.4P3, NI-502.31B6, and NI-502.8H1 for binding to the epitopes mentioned in Table I. Preferably however, the antibody of the present invention comprises in one or both of its immunoglobulin chains one, two or all three CDRs of the variable regions as set forth in FIG. 1 or one, two or all three CDRs which are 90%, 91%, 92%, 93%, 94%, 95%, 96%, 97%, 98% or 99% identical to the CDRs of the variable regions as set forth in FIG. 1. In addition, or alternatively, one or more framework regions (FRs) from the FRs are 80% identical to the corresponding FRs depicted in FIGS. 1A, B and C, respectively, preferably 85%, 90%, 95%, 96, 97%, 98%, 99% or 100% identical to the FRs depicted in FIGS. 1A, B and C, respectively. In some embodiments, 1, 2, 3, or all 4 FRs (each being at least 90%, 90-95%, and/or 95-99% identical to the FRs shown in FIGS. 1A, B and C, respectively is/are present.

As known in the art, CDR3 of the variable heavy chain (VH-CDR3) seems to mainly determine antigen specificity; see, e.g., Xu and Davis, Immunity 13 (2000), 37-45. In this context, it was noted that it is the diversity of heavy-chain CDR3s that drive specificity, whereas VH-CDR1 and VH-CDR2 residues are broadly cross-reactive and subject to improvement by somatic hypermutation; see Davis, Semin. Immunol. 16 (2004), 239-243. Accordingly, in one embodiment the antibody of the present invention, which has the immunological characteristics of any of the reference antibodies and being capable of competing with their binding to tau at the respective epitope comprise in their variable region at least VH-CDR3 of the corresponding reference antibody or a VH-CDR3 which amino acid sequence is at least 90% identical to the reference VH-CDR3, preferably 95% identical and more preferably 96%, 97%, 98%, 99% or 100% identical. For example, a variant antibody of a reference antibody may retain VH-CDR3 of the reference (parent) antibody while VH-CDR1 and/or VH-CDR2 may contain one or more amino acid substitutions; see supra.

In a further additional or alternative embodiment of the present invention the anti-Tau antibody, antigen-binding fragment, synthetic or biotechnological variant thereof can be optimized to have appropriate binding affinity to the target and stability properties. Therefore, at least one amino acid in the CDR or variable region, which is prone to modifications selected from the group consisting of glycosylation, oxidation, deamination, peptide bond cleavage, iso-aspartate formation and/or unpaired cysteine is substituted by a mutated amino acid that lacks such alteration or wherein at least one carbohydrate moiety is deleted or added chemically or enzymatically to the antibody, see, e.g. Liu et al., J. Pharm. Sci. 97 (2008), 2426-2447; Beck et al., Nat. Rev. Immunol. 10 (2010), 345-352; Haberger et al., MAbs. 6 (2014), 327-339.

An immunoglobulin or its encoding cDNA may be further modified. Thus, in a further embodiment, the method of the present invention comprises any one of the step(s) of producing a chimeric antibody, murinized antibody, single-chain antibody, Fab-fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor (1988) First edition; Second edition by Edward A. Greenfield, Dana-Farber Cancer Institute © 2014, ISBN 978-1-936113-81-1. For example, Fab and F(ab′)2 fragments may be produced recombinantly or by proteolytic cleavage of immunoglobulin molecules, using enzymes such as papain (to produce Fab fragments) or pepsin (to produce F(ab′)2 fragments). F(ab′)2 fragments contain the variable region, the light chain constant region (CL) and the CH1 domain of the heavy chain. Such fragments are sufficient for use, for example, in immunodiagnostic procedures involving coupling the immunospecific portions of immunoglobulins to detecting reagents such as radioisotopes.

In one embodiment, the antibody of the present invention may thus be provided in a format selected from the group consisting of a single chain Fv fragment (scFv), an F(ab′) fragment, an F(ab) fragment, and an F(ab′)₂ fragment, an Fd, an Fv, a single-chain antibody, and a disulfide-linked Fv (sdFv) and/or which is a chimeric murine-human or a murinized antibody.

As mentioned above, tauopathies usually come along with hyperphosphorylated tau as intracellular neurofibrillary tangles. Thus, in one embodiment, it may be beneficial to use recombinant Fab (rFab) and scFvs of the anti-tau antibody, which might more readily penetrate a cell membrane. For example, Krishnaswamy et al., J Neurosci. 34 (2014), 16835-16850 describe the use of smaller antibody fragments that bind to tau as being attractive as ligands for in vivo imaging and showed that peripheral injection of scFvs resulted in a strong in vivo brain signal in transgenic tauopathy mice, but not in wild-type or amyloid-β plaque mice. Furthermore, Nisbet et al. Brain 140 (2017), 1220-1230 demonstrated that intravenous administration of anti-tau scFvs to transgenic mice reduced anxiety-like behavior and tau hyperphosphorylation.

Immunotherapy approaches using different antibody formats such as scFv, single-domain antibody fragments (VHHs or sdAbs), bispecific antibodies, intrabodies and nanobodies have shown therapeutic efficacy in several animal models of Alzheimer's disease (AD), Parkinson disease (PD), dementia with Lewy bodies (DLB), frontotemporal dementia (FTD), Huntington disease (HD), transmissible spongiform encephalopathies (TSEs) and multiple sclerosis (MS). It has been demonstrated that recombinant antibody fragments may neutralize toxic extra- and intracellular misfolded proteins involved in the pathogenesis neurodegenerative diseases and thus represent a promising tool for the development of antibody-based immunotherapeutics for those diseases; see review of Manoutcharian et al., Curr Neuropharmacol 15 (2017), 779-788. The perceived advantages of using small Fab and scFv engineered antibody formats which lack the effector function include more efficient passage across the blood-brain barrier and minimizing the risk of triggering inflammatory side reactions. Furthermore, besides scFv and single-domain antibodies retain the binding specificity of full-length antibodies, they can be expressed as single genes and intracellularly in mammalian cells as intrabodies, with the potential for alteration of the folding, interactions, modifications, or subcellular localization of their targets; see for review, e.g., Miller and Messer, Molecular Therapy 12 (2005), 394-401.

However, as illustrated in the Examples in accordance with the present invention preferably complete IgG antibodies are used.

The five primary classes of immunoglobulins are IgG, IgM, IgA, IgD and IgE. These are distinguished by the type of heavy chain found in the molecule. IgG molecules have heavy chains known as gamma-chains; IgMs have mu-chains; IgAs have alpha-chains; IgEs have epsilon-chains; and IgDs have delta-chains; see for review, e.g., Schroeder et al., Structure and function of immunoglobulins. J. Allergy Clin. Immunol. 125 (2010), S41-S52. In principle, the antibodies of the present invention may be of any kind of class and antibody fragment as long as the binding specificity towards Tau as indicated in Table I and illustrated in the appended Examples for the corresponding reference antibody remains unaffected in kind. However, preferably complete IgG antibodies are used, wherein the antibody comprises a constant domain. The constant domain may be native, i.e. originally cloned together with the variable domain or heterologous, for example, a murine constant in case animal studies are envisaged. Preferably, the constant domain is of human origin with a different IgG subtype, e.g. IgG1 versus IgG4 or a different allotype and allele, respectively, compared to the constant domain of the antibody as naturally occurred in human. The definition of “allotypes” requires that antibody reagents are available to determine the allotypes serologically. If the determination is only done at the sequence level, the polymorphisms have to be described as “alleles”. This does not hinder to establish a correspondence with allotypes if the correspondence allele-allotype has been experimentally proven, or if the individual sequence is identical to a sequence for which it has been demonstrated.

In a preferred embodiment of the present invention, the constant domain is heterologous to at least one of the CDRs and the VH and VL chains, respectively, e.g. an immunoglobulin heavy chain constant domain and/or immunoglobulin light chain constant domain, preferably of the IgG type. In addition, or alternatively, the heterologous part of the antibody may be a mammalian secretory signal peptide. Put in other words, in one embodiment the anti-Tau antibody and Tau binding fragment, synthetic derivative, and biotechnological derivative thereof of the present invention is a (i) fusion protein comprising a polypeptide sequence which is heterologous to the VH region and/or VL region, or at least one CDR; and/or (ii) a non-natural variant of a polypeptide derived from an immunoglobulin, said non-natural variant comprising a heavy chain constant region that comprises one or more amino acid deletions, substitutions, and/or additions relative to a wild type polypeptide. For example, the human constant domain of the recombinant human-derived antibody of the present invention may be of a different IgG isotype than the constant domain of the parent antibody as naturally produced by the memory B cell or of a different allotype, for example to avoid or reduce immunogenicity which can happen as a result of allo-immunization; see, e.g., for review Jefferis and Lefranc, MAbs 1 (2009), 332-338.

As mentioned, five immunoglobulin isotypes exist, of which immunoglobulin G (IgG) is most abundant in human serum. Preferably, the immunoglobulin heavy and/or light chain constant domain present in the antibody of the present invention is of the IgG type.

The four subclasses, IgG1, IgG2, IgG3, and IgG4, which are highly conserved, differ in their constant region, particularly in their hinges and upper CH2 domains. These regions are involved in binding to both IgG-Fc receptors (FcgR) and C1q. As a result, the different subclasses have different effector functions, both in terms of triggering FcgR-expressing cells, resulting in phagocytosis or antibody-dependent cell-mediated cytotoxicity, and activating complement. The Fc regions also contain a binding epitope for the neonatal Fc receptor (FcRn), responsible for the extended half-life, placental transport, and bidirectional transport of IgG through mucosal surfaces. However, FcRn is also expressed in myeloid cells, where it participates in both phagocytosis and antigen presentation together with classical FcgR and complement. How these properties, IgG-polymorphisms and post-translational modification of the antibodies in the form of glycosylation, affect IgG-function is described in Vidarsson et al., (2014) IgG subclasses and allotypes: from structure to effector function. Front. Immunol. 5:520. doi:10.3389/fimmu.2014.00520 and de Taeye et al., Antibodies 2019, 8, 30; doi:10.3390/antib8020030. Preferably, the immunoglobulin heavy and/or light chain constant domain present in the antibody of the present invention is of the IgG type.

Accordingly, in certain embodiments of the present invention a specific IgG type is preferred, for example the IgG4 or IgG1 isotype and/or the constant region of the antibody, or antigen-binding fragment, variant, or derivative thereof has been altered so as to provide desired biochemical characteristics. In particular, in one embodiment the Fc portion of the antibody may be mutated to alter, i.e. to decrease or increase immune effector function or to increase its half-life using techniques known in the art. Thus, in one embodiment the Fc portion of the antibody is mutated to decrease immune effector function and in another embodiment the Fc portion of the antibody is mutated to increase immune effector function. In another embodiment, the antibody is mutated to increase its half-life.

For example, it may be that constant region modifications consistent with the instant invention moderate complement binding and thus reduce the serum half-life and nonspecific association of a conjugated cytotoxin. Other modifications of the constant region may be used to modify disulfide linkages or oligosaccharide moieties that allow for enhanced tissue antigen interaction due to increased antigen specificity or antibody flexibility. Furthermore, mutations in the Fc region can be made that lead to enhanced antibody dependent cell-mediated cytotoxicity (ADCC) or antibody-dependent cellular phagocytosis (ADCP) via increasing FcγRIIIa binding and/or decreasing FcγRIIIb binding and via increasing FcγRIIa binding and/or FcγRIIIa binding, respectively. For example, the GASDALIE Fc mutant (G236A/S239D/A330L/I332E) exhibits a higher affinity for FcγRIIIa. Another possibility is the enhancement of complement-dependent cytotoxicity (CDC) via increasing C1q binding and/or hexamerization.

In other embodiments, certain antibodies for use in the diagnostic and treatment methods described herein have a constant region, e.g., an IgG heavy chain constant region, which is altered to eliminate glycosylation, referred to elsewhere herein as aglycosylated or “agly” antibodies. Such “agly” antibodies may be prepared enzymatically as well as by engineering the consensus glycosylation site(s) in the constant region. It is believed that “agly” antibodies have a reduced effector function and thus an improved safety and stability profile in vivo.

Methods of producing aglycosylated antibodies, having desired effector function are found for example in international application WO 2005/018572, which is incorporated by reference in its entirety. A further approach to reduce the effector function of antibodies is the reduction of FcγR and C1q binding by mutations in the Fc region.

A summary is for example given in the review of Wang et al., Protein Cell 9 (2018), 63-73, wherein Table 1 provides examples of modifications to modulate antibody effector function and the half-life of an antibody and which mutations described therein are herein incorporated by reference. The resulting physiological profile, bioavailability and other biochemical effects of the modifications, such as recognizing and binding Tau, including pathologically hyperphosphorylated forms of Tau in dystrophic neurites, neurofibrillary tangles and neuropil threads in an immunohistochemical (IHC) assay with brain tissue of patients with AD, PSP and/or PiD and capturing Tau and AD-associated Tau in an immunoprecipitation assay with brain extracts of patients with AD, biodistribution and serum half-life, may easily be measured and quantified using well know immunological techniques without undue experimentation.

As mentioned, in some instances inflammatory responses should be avoided for which reason effector functions of the constant domain of the antibody may be attenuated or eliminated altogether. For example, recombinant human IgG antibodies (hIgGs) completely devoid of binding to Fcγ receptors (FcγRs) and complement protein C1q, and thus with abolished immune effector functions, are of use for various therapeutic applications. It was found that the combination of Leu234Ala and Leu235Ala (commonly called LALA mutations) or the SPLE mutation eliminated FcγRIIa binding and were shown to eliminate detectable binding to FcγRI, Ha, and Ma for both IgG1 and IgG4 and that the LALA-PG mutation was an improvement over LALA mutations alone in that they nullified Fc function in mouse and human IgG; for corresponding review see, e.g., Saunders (2019) Conceptual Approaches to Modulating Antibody Effector Functions and Circulation Half-Life. Front. Immunol. 10:1296.doi: 10.3389/fimmu.2019.01296 and Schlothauer et al., Protein Engineering, Design and Selection 29 (2016), 457-466.

Another early approach to reduce effector function is to mutate the glycosylation site at N297 with mutations such as N297A, N297Q, and N297G. The half-life of an antibody can be increased via introducing the following mutations M252Y/S254T/T256E or M428L/N434S; see Wang et al. 2018.

In this context, Lee et al., Cell Rep 16 (2016), 1690-1700 showed that antibody effector function is not essential for targeting tau since the antibodies, one with and the other without effector function, reduced accumulation of tau pathology in Tau-P301L transgenic mice and protected cultured neurons against extracellular tau-induced toxicity. Thus, in one embodiment the antibody is of the IgG1 class or isotype preferably, wherein the antibody is an IgG1 variant comprising the amino acid substitutions L234A, L235A (LALA) and preferably the amino acid substitutions L234A, L235A, P329G (LALA-PG). For example, in order to avoid recruitment of immune cells through Fcγ-receptors and enable a short systemic half-life in circulation, FcγR binding can be abolished by introduction of P329G LALA mutations, see Schlothauer et al. (2016), supra, while FcRn binding and recycling can be abolished by introduction of Triple A (I253A, H310A, H435A) mutations; see, e.g., Regula et al., EMBO Mol. Med., 8 (2016), 1265-1288.

Furthermore, human immunoglobulin G isotype 4 (IgG4) antibodies are potential candidates for antibody therapy when reduced immune effector functions are desirable. Thus, in another embodiment, the antibody is of the IgG4 class or isotype. IgG4 antibodies are dynamic molecules able to undergo a process known as Fab arm exchange (FAE). This results in functionally monovalent, bispecific antibodies (bsAbs) with unknown specificity and hence, potentially, reduced therapeutic efficacy. Thus, the antibody of the present invention is of the IgG4 class or isotype including the S228P mutation. The S228P mutation prevents in vivo and in vitro IgG4 Fab-arm exchange as demonstrated using a combination of novel quantitative immunoassays and physiological matrix preparation; see Silva et al., J. Biol. Chem. 290 (2015), 5462-5469.

The present invention also relates to one or more polynucleotide(s) encoding the antibody or antigen-binding fragment thereof of the present invention or an immunoglobulin VH and VL thereof, preferably wherein the polynucleotide(s) is (are) cDNA.

In a preferred embodiment of the present invention, the polynucleotide comprises, consists essentially of, or consists of a nucleic acid having a polynucleotide sequence encoding the VH or VL chain of an anti-Tau antibody as depicted in Table III. In this respect, the person skilled in the art will readily appreciate that the polynucleotides encoding the light and/or heavy chain may be encoded by one or more polynucleotides. In one embodiment therefore, the polynucleotide comprises, consists essentially of, or consists of a nucleic acid having a polynucleotide sequence of the V_{H} and the V_{L} chain of an anti-Tau antibody as depicted in Table III.

TABLE III
Nucleotide and amino acid sequences of the variable regions (VH, VL) of the
antibodies NI-502.4P3, NI-502.31B6, and NI-502.8H1 of the present invention.
Underlined, bold nucleotides or amino acids indicate the CDR coding regions
in the variable chain sequence.

| | Nucleotide and amino acid sequence of the |
|---|---|
| Antibody | variable heavy (VH) and variable light (VL) chains. |
| NI-502.4P3- | gaggtgcagctggtgcagtctggagcagaggtgaagaagcccggggagtctctg |
| VH | aagatctcctgtaaggtttctggatacgactttagcaagtactggatcggctgg |
| | gtgcgccagatgcccgggaaaggcctggagtggatggggctcatctatcctggt |
| | gactctgataccagatatagtccatctttggaagggcaagtcagcatctcagcc |
| | gacaagtccatcagcgccgcctacttgcaattcagcagcctgaaggcctcggac |
| | agcgccatgtattattgtgtgaggaagggatatagtgcctaccttggagggtgg |
| | ttcgacccctggggccagggaaccctggtcaccgtctcctcg |
| | SEQ ID NO: 1 |
| NI-502.4P3- | EVQLVQSGAEVKKPGESLKISCKVSGYDFSKYWIGWVRQMPGKGLEWMGLIYPG |
| VH | DSDTRYSPSLEGQVSISADKSISAAYLQFSSLKASDSAMYYCVRKGYSAYLGGW |
| | FDPWGQGTLVTVSS SEQ ID NO: 2 |
| NI-502.4P3- | gacatccagttgacccagtctccagactccctggctgcgtctctgggcgagagg |
| VL | gtcaccatcaactgcaggtccagccagaatattttttacaactccaacaataag |
| | aacttcttagcttggtatcagcagaaagcaggacagtctcctaagttgcttatt |
| | tactgggcttctacgcgggaatccggggtccctgaccgattcagtggcagcggc |
| | tctgggacagatttcactctcaccatcagcggcctgcaggctggagatgtggca |
| | gtttatttctgtcagcaatattttactgatgctcccactttcggcggagggacc |
| | aaggtggagatcaaa SEQ ID NO: 6 |
| NI-502.4P3- | DIQLTQSPDSLAASLGERVTINCRSSQNIFYNSNNKNFLAWYQQKAGQSPKLLI |
| VL | YWASTRESGVPDRFSGSGSGTDFTLTISGLQAGDVAVYFCQQYFTDAPTFGGGT |
| | KVEIK SEQ ID NO: 7 |
| NI-502.31B6- | gaggtgcagctgttgcagtctgggggaggcgtggtccagcctggggggtccctg |
| VH | agactctcctgtgaagcctctggattcaccttcagttcctattctatgcactgg |
| | gtccgccaggctccaggcaaggggctggaatggctgacacttatctcatataat |
| | ggagtcaataagtattacgcaaagtccgtgaagggccgattcgccatctccaga |
| | gacgattccaagagcacactgtttttgcaaatgaacagcctgacaactgacgac |
| | acggctgtatattactgtgcgagatgtccaatcccgcgggcggcgtcgtataga |
| | aatggagataattatttcgaattcggccagggcaccctggtcaccgtctcctcg |
| | SEQ ID NO: 11 |
| NI-502.31B6- | EVQLLQSGGGVVQPGGSLRLSCEASGFTFSSYSMHWVRQAPGKGLEWLTLISYN |
| VH | GVNKYYAKSVKGRFAISRDDSKSTLFLQMNSLTTDDTAVYYCARCPIPRAASYR |
| | NGDNYFEFGQGTLVTVSS SEQ ID NO: 12 |
| NI-502.31B6- | caggctgtggtgactcaggagccatcgctctcagtgtcccctggagggacagtc |
| VL | acactcacttgtgccctgagttctgcctcagtctctacttcttcgtaccccagc |
| | tggtaccagcagaccccaggccagcctccacgcatgctcatccacaacacagac |
| | actcgctcttctggtgtccctgatcgcttctctggctccatccttgggaagaaa |
| | gctgccctcaccatcacgggggcccaggcagatgatgaatctgattactactgt |
| | gtggtatatttggggaatggcttttgggtgttcggcggagggaccaagctgacc |
| | gtccta SEQ ID NO: 16 |
| NI-502.31B6- | QAVVTQEPSLSVSPGGTVTLTCALSSASVSTSSYPSWYQQTPGQPPRMLIHNTD |
| VL | TRSSGVPDRFSGSILGKKAALTITGAQADDESDYYCVVYLGNGFWVFGGGTKLT |
| | VL SEQ ID NO: 17 |
| NI-502.8H1- | gaggtgcagctggtggagtctgggggaggcttggtgcagccgggggggtcccta |
| VH | agactctcctgtgcagcctccggattcacctttagcatctataccatgagctgg |
| | gtccgccaggctccagggaaggggctggagtgggtctcagctattctcagtgat |
| | ggtagcacatactatgcagactccgtagagggccgattcaccatctccagagac |
| | aattccaagaccaccctgtatctacaaatgaacagcctgacagccgaggacacg |
| | gccgtatattattgtacaaaacgtgttgaggggagctttcgggggtggggccag |
| | ggaaccctggtcaccgtctcctcg SEQ ID NO: 21 |
| NI-502.8H1- | EVQLVESGGGLVQPGGSLRLSCAASGFTFSIYTMSWVRQAPGKGLEWVSAILSD |
| VH | GSTYYADSVEGRFTISRDNSKTTLYLQMNSLTAEDTAVYYCTKRVEGSFRGWGQ |
| | GTLVTVSS SEQ ID NO: 22 |
| NI-502.8H1- | gaaattgtgatgacacagtctccaggcaccctgtctttgtctccaggggaaagc |
| VL | gccaccctctcctgcagggccagtcagagtgttagcactacctacttagcctgg |
| | taccagcagaaacctggccaggctcccaggctcctcatctatggtgcatccagc |
| | agggccactggcatcccagacagattcagtggcagtgggtctgggacagacttc |
| | actctcacaatcagcagactggagcctgaagattttgcagtgtattactgtcag |
| | cagtatggtgggtcacgcttgatcaccttcggccaagggacacgactggagatt |
| | aaa SEQ ID NO: 26 |
| NI-502.8H1- | EIVMTQSPGTLSLSPGESATLSCRASQSVSTTYLAWYQQKPGQAPRLLIYGASS |
| VL | RATGIPDRFSGSGSGTDFTLTISRLEPEDFAVYYCQQYGGSRLITFGQGTRLEI |
| | K SEQ ID NO: 27 |

In one embodiment of the present invention, the polynucleotide(s) are linked to a heterologous nucleic acid, for example expression control sequences such as a promoter, transcription and/or translation enhancer sequences, internal ribosome binding sites, nucleic acids encoding a peptide leader sequence for recombinant expression in a host and the like. Accordingly, the present invention relates to a polynucleotide encoding a human-derived recombinant anti-Tau antibody or Tau binding fragment, synthetic derivative, or biotechnological derivative thereof, wherein the polynucleotide encodes
(i) a VH chain comprising CDRs 1, 2, and 3, and/or a VL chain comprising VL CDRs 1, 2, and 3 as defined by Kabat, wherein
   (a) VH-CDR1 comprises the amino acid sequence of SEQ ID NO: 3 or a variant thereof, wherein the variant comprises one or two amino acid substitutions,
   (b) VH-CDR2 comprises the amino acid sequence of SEQ ID NO: 4 or a variant thereof, wherein the variant comprises one or two amino acid substitutions,
   (c) VH-CDR3 comprises the amino acid sequence of SEQ ID NO: 5 or a variant thereof, wherein the variant comprises one or two amino acid substitutions,
   (d) VL-CDR1 comprises the amino acid sequence of SEQ ID NO: 8 or a variant thereof, wherein the variant comprises one or two amino acid substitutions,
   (e) VL-CDR2 comprises the amino acid sequence of SEQ ID NO: 9 or a variant thereof, wherein the variant comprises one or two amino acid substitutions, and
   (f) VL-CDR3 comprises the amino acid sequence of SEQ ID NO: 10 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 2 or a variant thereof, wherein the variant comprises one or more amino acid substitutions; and
   (b) the VL comprises the amino acid sequence depicted in SEQ ID NO: 7, or a variant thereof, wherein the variant comprises one or more amino acid substitutions;
   preferably wherein the VH and VL chain amino acid sequence is at least 90% identical to SEQ ID NO: 2 and 7, respectively; or
(iii) a VH chain comprising CDRs 1, 2, and 3, and/or a VL chain comprising VL CDRs 1, 2, and 3 as defined by Kabat, wherein
   (a) VH-CDR1 comprises the amino acid sequence of SEQ ID NO: 13 or a variant thereof, wherein the variant comprises one or two amino acid substitutions,
   (b) VH-CDR2 comprises the amino acid sequence of SEQ ID NO: 14 or a variant thereof, wherein the variant comprises one or two amino acid substitutions,
   (c) VH-CDR3 comprises the amino acid sequence of SEQ ID NO: 15 or a variant thereof, wherein the variant comprises one or two amino acid substitutions,
   (d) VL-CDR1 comprises the amino acid sequence of SEQ ID NO: 18 or a variant thereof, wherein the variant comprises one or two amino acid substitutions,
   (e) VL-CDR2 comprises the amino acid sequence of SEQ ID NO: 19 or a variant thereof, wherein the variant comprises one or two amino acid substitutions, and
   (f) VL-CDR3 comprises the amino acid sequence of SEQ ID NO: 20 or a variant thereof, wherein the variant comprises one or two amino acid substitutions; and/or
(iv) a VH chain and/or a VL chain, wherein
   (a) the VH chain comprises the amino acid sequence depicted in SEQ ID NO: 12 or a variant thereof, wherein the variant comprises one or more amino acid substitutions; and
   (b) the VL comprises the amino acid sequence depicted in SEQ ID NO: 17, or a variant thereof, wherein the variant comprises one or more amino acid substitutions;
   preferably wherein the VH and VL chain amino acid sequence is at least 90% identical to SEQ ID NO: 12 and 17, respectively; or
(v) a VH chain comprising CDRs 1, 2, and 3, and/or a VL chain comprising VL CDRs 1, 2, and 3 as defined by Kabat, wherein
   (a) VH-CDR1 comprises the amino acid sequence of SEQ ID NO: 23 or a variant thereof, wherein the variant comprises one or two amino acid substitutions,
   (b) VH-CDR2 comprises the amino acid sequence of SEQ ID NO: 24 or a variant thereof, wherein the variant comprises one or two amino acid substitutions,
   (c) VH-CDR3 comprises the amino acid sequence of SEQ ID NO: 25 or a variant thereof, wherein the variant comprises one or two amino acid substitutions,
   (d) VL-CDR1 comprises the amino acid sequence of SEQ ID NO: 28 or a variant thereof, wherein the variant comprises one or two amino acid substitutions,
   (e) VL-CDR2 comprises the amino acid sequence of SEQ ID NO: 29 or a variant thereof, wherein the variant comprises one or two amino acid substitutions, and
   (f) VL-CDR3 comprises the amino acid sequence of SEQ ID NO: 30 or a variant thereof, wherein the variant comprises one or two amino acid substitutions; and/or
(vi) a VH chain and/or a VL chain, wherein
   (a) the VH chain comprises the amino acid sequence depicted in SEQ ID NO: 22 or a variant thereof, wherein the variant comprises one or more amino acid substitutions; and
   (b) the VL comprises the amino acid sequence depicted in SEQ ID NO: 27, or a variant thereof, wherein the variant comprises one or more amino acid substitutions;
   preferably wherein the VH and VL chain amino acid sequence is at least 90% identical to SEQ ID NO: 22 and 27, respectively.

In addition, the present invention relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 3, 4, and 5, respectively, and wherein the VH when paired with a light chain variable region (VL) comprising the amino acid sequence set forth in SEQ ID NO: 7 binds to Tau;
(b) a polynucleotide encoding an immunoglobulin light chain or a fragment thereof comprising a VL comprising CDRs 1, 2, and 3 with the amino acid sequences set forth in SEQ ID NOs: 8, 9, and 10, respectively, and wherein the VL when paired with a VH comprising the amino acid sequence set forth in SEQ ID NO: 2 binds to Tau;
(c) a polynucleotide encoding
   (i) an immunoglobulin heavy chain or a fragment thereof comprising a VH comprising CDRs 1, 2, and 3 with the amino acid sequences set forth in SEQ ID NOs: 3, 4, and 5, respectively; and
   (ii) an immunoglobulin light chain or a fragment thereof comprising a VL comprising CDRs 1, 2, and 3 with the amino acid sequences set forth in SEQ ID NOs: 8, 9, and 10, respectively;
(d) a polynucleotide encoding an immunoglobulin heavy chain or a fragment thereof comprising a VH comprising the amino acid sequence set forth in SEQ ID NO: 2, wherein the VH when paired with a VL comprising the amino acid sequence set forth in SEQ ID NO: 7 binds to Tau;
(e) a polynucleotide encoding an immunoglobulin light chain or a fragment thereof comprising a VL comprising the amino acid sequence set forth in SEQ ID NO: 7, wherein the VL when paired with a VH comprising the amino acid sequence set forth in SEQ ID NO: 2 binds to Tau;
(f) a polynucleotide encoding an immunoglobulin heavy chain or a fragment thereof comprising a VH comprising the amino acid sequence set forth in SEQ ID NO: 2 and an immunoglobulin light chain or a fragment thereof comprising a VL comprising the amino acid sequence set forth in SEQ ID NO: 7;
(g) a polynucleotide as in any one of (a)-(f), wherein a CDR comprises one or more, preferably no more than two amino acid substitution and/or the variable region sequence is at least 90% identical to SEQ ID NO: 2 or SEQ ID NO: 7.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 13, 14, and 15, respectively, and wherein the VH when paired with a light chain variable region (VL) comprising the amino acid sequence set forth in SEQ ID NO: 17 binds to Tau;
(b) a polynucleotide encoding an immunoglobulin light chain or a fragment thereof comprising a VL comprising CDRs 1, 2, and 3 with the amino acid sequences set forth in SEQ ID NOs: 18, 19, and 20, respectively, and wherein the VL when paired with a VH comprising the amino acid sequence set forth in SEQ ID NO: 12 binds to Tau;
(c) a polynucleotide encoding
   (i) an immunoglobulin heavy chain or a fragment thereof comprising a VH comprising CDRs 1, 2, and 3 with the amino acid sequences set forth in SEQ ID NOs: 13, 14, and 15, respectively; and
   (ii) an immunoglobulin light chain or a fragment thereof comprising a VL comprising CDRs 1, 2, and 3 with the amino acid sequences set forth in SEQ ID NOs: 18, 19, and 20, respectively;
(d) a polynucleotide encoding an immunoglobulin heavy chain or a fragment thereof comprising a VH comprising the amino acid sequence set forth in SEQ ID NO: 12, wherein the VH when paired with a VL comprising the amino acid sequence set forth in SEQ ID NO: 17 binds to Tau;
(e) a polynucleotide encoding an immunoglobulin light chain or a fragment thereof comprising a VL comprising the amino acid sequence set forth in SEQ ID NO: 17, wherein the VL when paired with a VH comprising the amino acid sequence set forth in SEQ ID NO: 12 binds to Tau;
(f) a polynucleotide encoding an immunoglobulin heavy chain or a fragment thereof comprising a VH comprising the amino acid sequence set forth in SEQ ID NO: 12 and an immunoglobulin light chain or a fragment thereof comprising a VL comprising the amino acid sequence set forth in SEQ ID NO: 17;
(g) a polynucleotide as in any one of (a)-(f), wherein a CDR comprises one or more, preferably no more than two amino acid substitution and/or the variable region sequence is at least 90% identical to SEQ ID NO: 12 or SEQ ID NO: 17.

Alternatively, the present invention relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 23, 24, and 25, respectively, and wherein the VH when paired with a light chain variable region (VL) comprising the amino acid sequence set forth in SEQ ID NO: 27 binds to Tau;
(b) a polynucleotide encoding an immunoglobulin light chain or a fragment thereof comprising a VL comprising CDRs 1, 2, and 3 with the amino acid sequences set forth in SEQ ID NOs: 28, 29, and 30, respectively, and wherein the VL when paired with a VH comprising the amino acid sequence set forth in SEQ ID NO: 22 binds to Tau;
(c) a polynucleotide encoding
   (i) an immunoglobulin heavy chain or a fragment thereof comprising a VH comprising CDRs 1, 2, and 3 with the amino acid sequences set forth in SEQ ID NOs: 23, 24, and 25, respectively; and
   (ii) an immunoglobulin light chain or a fragment thereof comprising a VL comprising CDRs 1, 2, and 3 with the amino acid sequences set forth in SEQ ID NOs: 28, 29, and 30, respectively;
(d) a polynucleotide encoding an immunoglobulin heavy chain or a fragment thereof comprising a VH comprising the amino acid sequence set forth in SEQ ID NO: 22, wherein the VH when paired with a VL comprising the amino acid sequence set forth in SEQ ID NO: 27 binds to Tau;
(e) a polynucleotide encoding an immunoglobulin light chain or a fragment thereof comprising a VL comprising the amino acid sequence set forth in SEQ ID NO: 27, wherein the VL when paired with a VH comprising the amino acid sequence set forth in SEQ ID NO: 22 binds to Tau;
(f) a polynucleotide encoding an immunoglobulin heavy chain or a fragment thereof comprising a VH comprising the amino acid sequence set forth in SEQ ID NO: 22 and an immunoglobulin light chain or a fragment thereof comprising a VL comprising the amino acid sequence set forth in SEQ ID NO: 27;
(g) a polynucleotide as in any one of (a)-(f), wherein a CDR comprises one or more, preferably no more than two amino acid substitution and/or the variable region sequence is at least 90% identical to SEQ ID NO: 22 or SEQ ID NO: 27.

In a preferred embodiment, the immunoglobulin of any of the preceding paragraphs when paired as VH and VL binds to Tau in diseased human brain tissue. In addition, or alternatively the immunoglobulin of any of the preceding paragraphs when paired as VH and VL binds to pathological hyperphosphorylated Tau. Furthermore, the immunoglobulin of any of the preceding paragraphs may further or alternatively when paired as VH and VL bind Tau as measured by indirect ELISA or an equivalent assay to the assay described in the Examples.

Furthermore, the present invention relates to a vector and vectors comprising one or more of those polynucleotides, preferably wherein the vector is an expression vector and the one or more polynucleotide(s) are operably linked to expression control sequences.

The polynucleotides may be produced and, if desired manipulated using methods well known in the art for the manipulation of nucleotide sequences, e.g., recombinant DNA techniques, site directed mutagenesis, PCR, etc. (see, for example, the techniques described in Molecular Cloning: A Laboratory Manual (Fourth Edition): Three-volume set; Green and Sambrook (2012) ISBN 10: 1936113422/ISBN 13: 9781936113422 Cold Spring Harbor Laboratory Press; update (2014) ISBN 978-1-936113-42-2 and Ausubel et al., eds., Current Protocols in Molecular Biology, John Wiley & Sons, NY (1998) and updates, which are both incorporated by reference herein in their entireties), to generate antibodies having a different amino acid sequence, for example to create amino acid substitutions, deletions, and/or insertions.

Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operable linked to a promoter. Such vectors may include the nucleotide sequence encoding the constant region of the antibody molecule (see, e.g., international applications WO 86/05807 and WO 89/01036; and U.S. Pat. No. 5,122,464) and the variable domain of the antibody may be cloned into such a vector for expression of the entire heavy or light chain.

The term “vector” or “expression vector” is used herein to mean vectors used in accordance with the present invention as a vehicle for introducing into and expressing a desired gene in a host cell. In general, vectors compatible with the instant invention will comprise a selection marker, appropriate restriction sites to facilitate cloning of the desired gene and the ability to enter and/or replicate in eukaryotic or prokaryotic cells. The marker may provide for prototrophy to an auxotrophic host, biocide resistance (e.g., antibiotics), or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. For the expression of double-chained antibodies, a single vector or vectors encoding both the heavy and light chains may be co-expressed in the host cell for expression of the entire immunoglobulin molecule, as detailed below.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain; see Proudfoot, Nature 322 (1986), 52; Kohler, Proc. Natl. Acad. Sci. USA 77 (1980), 2197. The coding sequences for the heavy and light chains may comprise cDNA or genomic DNA. The expression vector(s) is (are) transferred to a host cell by conventional techniques and the transfected cells are then cultured by conventional techniques to produce an antibody for use in the methods described herein. Accordingly, the present invention also relates to host cells comprising one or more polynucleotides or a vector or vectors of the present invention.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

Antibodies used for laboratory research/diagnosis may be expressed in any suitable host, e.g. in mammalian cells, bacterial cells, yeasts, plant cells or insect cells. However, currently almost all therapeutic antibodies are still produced in mammalian cell lines in order to reduce the risk of immunogenicity due to altered, non-human glycosylation patterns. However, recent developments of glycosylation-engineered yeast, insect cell lines, and transgenic plants are promising to obtain antibodies with “human-like” post-translational modifications. Furthermore, smaller antibody fragments including bispecific antibodies without any glycosylation are successfully produced in bacteria and have advanced to clinical testing. The first therapeutic antibody products from a non-mammalian source can be expected in coming next years. A review on current antibody production systems that can be applied for preparing the human-derived recombinant anti-Tau antibody or Tau binding fragment, synthetic derivative, or biotechnological derivative thereof of the present invention including their usability for different applications is given in Frenzel et al., Front Immunol. 4 (2013), 217, published online on Jul. 29, 2013 doi: 10.3389/fimmu.2013.00217 and transient expression of human antibodies in mammalian cells is described by Vazquez-Lombardi et al., Nature protocols 13 (2018), 99-117; and Hunter et al., Optimization of protein expression in mammalian cells. Current Protocols in Protein Science 95 (2019), e77. doi: 10.1002/cpps.77. Once an antibody molecule of the invention has been recombinantly expressed, the whole antibodies, their dimers, individual light and heavy chains, or other immunoglobulin forms of the present invention can be purified according to standard procedures of the art, including for example, by chromatography (e.g., ion exchange, affinity, particularly by affinity for the specific antigen after Protein A, and sizing column chromatography), centrifugation, differential solubility, e.g. ammonium sulfate precipitation, or by any other standard technique for the purification of proteins; see, e.g., Scopes, “Protein Purification”, Springer Verlag, N.Y. (1982) and Antibodies A Laboratory Manual 2nd edition, 2014 by Cold Spring Harbor Laboratory Press, Cold Spring Harbor, New York, USA. Thus, the present invention also relates to a method for preparing an anti-Tau antibody and/or fragments thereof or immunoglobulin chain(s) thereof, said method comprising:
(a) culturing the host cell as defined hereinabove, which cell comprised the polynucleotide(s) or vector(s) as defined hereinbefore under conditions allowing for expression of the anti-Tau antibody, Tau-binding fragment or immunoglobulin chain(s) thereof; and
(b) isolating the anti-Tau antibody, Tau-binding fragment or immunoglobulin chain(s) thereof from the culture.

Furthermore, the present invention also relates to the anti-Tau antibody, Tau-binding fragment and immunoglobulin chain(s) thereof encoded by a polynucleotide as defined hereinabove and/or obtainable by the method for their recombinant production mentioned above.

In one embodiment, the present invention relates to a method of diagnosing a tauopathic disease as defined hereinbefore, preferably AD, PSP and/or PiD in a subject, the method comprising determining the presence of Tau and/or pathologically modified Tau in a sample from the subject to be diagnosed with at least one antibody of the present invention or an Tau binding fragment thereof, wherein the presence of pathologically modified Tau is indicative of a neurodegenerative tauopathy and an increase of the level of the pathologically modified Tau in comparison to the level of the physiological Tau forms is indicative for progression of a neurodegenerative tauopathy in said subject. The subject is diagnosed with a tauopathy if the sample contains pathologically modified and/or aggregated tau, and subsequently the subject is administered an anti-tau antibody of the invention. Alternatively, the subject is diagnosed in accordance with the method of the present, the information is transmitted directly or indirectly to the subject or to a physician or medical institute, and if the subject has been diagnosed with a tauopathy the subject is treated with an agent which is capable of ameliorating, treating or reducing the progression of at least one symptom of the tauopathy in the subject. Preferably, the agent is an anti-tau antibody, most preferably an antibody of the present invention.

The subject to be diagnosed may be asymptomatic or preclinical for the disease. In one embodiment, the control subject has a tauopathic disease, for example, AD, amyotrophic lateral sclerosis-parkinsonism-dementia (ALS-PDC), argyrophilic grain disease (AGD), corticobasal degeneration (CBD), Creutzfeldt Jakob Disease (CJD), Frontotemporal dementia (FTD), Frontotemporal dementia with parkinsonism-17 (FTDP-17), Niemann-Pick disease, type C (NP-C), PiD, PSP or other tauopathies as mentioned below, wherein a similarity between the level of pathologically modified Tau and the reference standard indicates that the subject to be diagnosed has a tauopathic disease. Alternatively, or in addition as a second control the control subject does not have a tauopathic disease, wherein a difference between the level of Tau and/or of pathologically modified Tau and the reference standard indicates that the subject to be diagnosed has a tauopathic disease. In one embodiment, the subject to be diagnosed and the control subject(s) are age-matched. The sample to be analyzed may be any body fluid suspected to contain pathologically modified and/or aggregated tau, for example a blood, CSF, or urine sample.

The level of Tau and/or of pathologically modified Tau may be assessed by any suitable method known in the art comprising, e.g., analyzing Tau by one or more techniques chosen from Western blot, immunoprecipitation, enzyme-linked immunosorbent assay (ELISA), radioimmunoassay (MA), fluorescent activated cell sorting (FACS), two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), and laser densitometry.

Furthermore, the anti-Tau antibody or Tau-binding fragment thereof can be used for in vivo imaging of Tau. Thus, in one embodiment, said in vivo imaging of Tau comprises positron emission tomography (PET), single photon emission tomography (SPECT), near infrared (NIR) optical imaging or magnetic resonance imaging (MRI). In particular, the anti-Tau antibody or Tau-binding fragment thereof can be used for or is useful for Tau PET imaging of AD patients treated with anti-AB drugs, e.g. Aducanumab and Gantenerumab.

Methods of diagnosing a tauopathic disease such as Alzheimer's disease, monitoring a tauopathic disease progression, and monitoring a tauopathic disease treatment using antibodies and related means which may be adapted in accordance with the present invention are also described in international applications WO93/08302 A1, WO94/13795 A1, WO95/17429 A1, WO96/04309 A1, WO2002/062851 A1 and WO2004/016655 A1. Similarly, antibody-based detection methods for Tau are described in international application WO2005/080986, the disclosure content of all being incorporated herein by reference. Those methods may be applied as described but with a Tau specific antibody, binding fragment, derivative or variant of the present invention.

In certain embodiments, the antibody polypeptide comprises an amino acid sequence or one or more moieties not normally associated with an antibody. Thus, the present invention further encompasses antibodies, or antigen-binding fragments, variants, or derivatives thereof of the invention conjugated to a diagnostic or therapeutic agent. The antibodies can be used diagnostically to, for example, demonstrate presence of a neurological disease, to indicate the risk of getting a neurological disease, to monitor the development or progression of a neurological disease, i.e. tauopathic disease as part of a clinical testing procedure to, e.g., determine the efficacy of a given treatment and/or prevention regimen. Detection can be facilitated by coupling the antibody, or antigen-binding fragment, variant, or derivative thereof to a detectable substance. Exemplary modifications are described in more detail below. For example, the antibody or Tau-binding fragment thereof such a single-chain Fv antibody fragment of the invention may comprise a flexible linker sequence, or may be modified to add a functional moiety or detectable label (e.g., PEG, a drug, a toxin, or a label such as a fluorescent, (chemo/bio)luminescent, radioactive, enzyme, nuclear magnetic, heavy metal, a tag, a flag and the like); see, e.g., Antibodies A Laboratory Manual 2nd edition, 2014 by Cold Spring Harbor Laboratory Press, Cold Spring Harbor, New York, USA for general techniques; Dean and Palmer, Nat. Chem. Biol. 10 (2014), 512-523, for advances in fluorescence labeling strategies for dynamic cellular imaging; and Falck and Müller, Antibodies 7 (2018), 4; doi:10.3390/antib7010004 for enzyme-based labeling strategies for antibody-drug conjugates and antibody mimetics.

Furthermore, the anti-Tau antibody or Tau-binding fragments of the present invention can comprise a brain targeting entity and/or is contained in or conjugated to a vehicle such as an exosome or nanoparticle for delivery to the brain.

As it is well known in the art, the blood-brain barrier (BBB) restricts drug efficacy for central nervous system (CNS) diseases. For example, monoclonal antibodies do not cross the BBB efficiently, reaching a maximum of 0.11% at 1 hour after injection (Banks et al. (2002), Peptides 23, 2223-2226). The BBB is a specialized structural, physiological and biochemical barrier and serves as the first interface between the changeable environment of blood and the extracellular fluid in the CNS. The BBB regulates the homeostasis of the nervous system by strictly controlling the movement of small molecules or macromolecules from the blood to the brain. It only permits selective transport of molecules that are essential for brain function. In detail, more than 98% of small molecule drugs and almost 100% of large molecule drugs are precluded from drug delivery to brain (Redzic (2011) Fluids Barriers CNS 8, 3; Pardridge (2005) NeuroRx, 2, 3-14). Thus, the polypeptide and the antibody, antigen-binding fragment thereof, variant or derivative thereof, respectively may be modified in order to be able to penetrate the BBB.

For example, said antibodies and binding fragments can be fused to cell-penetrating peptides (CPPs), which qualify as brain targeting entity and which are usually short cationic and/or amphipathic peptides that have the ability to transport the associated molecular cargo (e.g., peptides, proteins, antibodies, etc.) across cellular membranes. However, also anionic CCPs have been reported. Examples are given in Sharma et al. (2016) Int. J. Mol. Sci. 17, 806 and instructions how to fuse an antibody with a CPP are for example provided in Gaston et al. (2019) Sci. Rep. 9, 18688 doi:10.1038/s41598-019-55091-0. Furthermore, polyamine modification has been shown to dramatically increase the penetration of i.a. antibodies across the BBB (Poduslo and Curran (1996) J. Neurochem. 66, 1599-1609). The most investigated method to deliver macromolecules into the brain is via receptor-mediated transcytosis (RMT) and the main RMT receptors that have been studied are the transferrin receptor (TfR) and insulin receptor (IR). Thus, RMT receptors are also brain targeting entities. For example, bispecific antibodies have emerged as promising scaffolds to deliver therapeutic antibodies to the brain via engineering the antibody to incorporate one arm with specificity against a BBB RMT receptor, which drives their transmission across the BBB, and the other arm against a CNS therapeutic agent. Essentially, bispecific antibodies can be generated by fusion of antibody fragments such as Fabs, scFv or single domain antibodies into the N- or C-terminal of a convention IgG molecule or by heterodimerization strategies such as the “knobs-into-holes” technology developed by Genentech; see for details Neves et al. (2016) Trends Biotech. 34, 36-48.

Thus, the anti-Tau antibody of the present invention can be a bispecific antibody binding to Tau and to a BBB RMT receptor.

In another approach, lipid nanoparticles/nanoexosomes can be used to deliver the antibodies or binding fragments of the present invention across the BBB. For example, dually decorated nanoliposomes with an anti-Tau monoclonal antibody and an anti-RMT antibody, e.g. anti-TfR monoclonal antibody using biotin streptavidin conjugation can be used for improved delivery across the blood brain barrier. This principle is outlined in Markoutsa et al. (2012) Eur. J. Pharm. Biopharm. 81, 49-56) with an anti-Aβ antibody instead of an anti-Tau antibody.

Furthermore, as summarized in Tosi et al. (2013) (Curr. Med. Chem. 20, 2212-25), biodegradable nanoparticles formulated from poly(D,L-lactide-co-glycolide) (PLGA) have been extensively investigated for sustained and targeted delivery of different agents, including antibodies across the BBB. Thus, the antibodies and binding fragments of the present invention are conjugated to nanoparticles and nanoexosomes, respectively.

Accordingly, in one embodiment, the anti-Tau antibody or Tau-binding fragment thereof of the present invention is capable to penetrate the BBB.

An antibody polypeptide of the invention may comprise, consist essentially of, or consist of a fusion protein. Fusion proteins are chimeric molecules which comprise, for example, an immunoglobulin Tau-binding domain with at least one target binding site, and at least one heterologous portion, i.e. a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an antibody, or an antigen-binding fragment, variant, or analog thereof is derived from a non-immunoglobulin polypeptide of the same species, or an immunoglobulin or non-immunoglobulin polypeptide of a different species.

The human-derived recombinant anti-Tau antibody or Tau-binding fragment, synthetic derivative, or biotechnological derivative thereof, optionally as fusion protein and/or labeled as described hereinbefore is then provided for various applications in accordance with standard techniques known in the art; see, e.g., Antibodies A Laboratory Manual 2nd edition, 2014 by Cold Spring Harbor Laboratory Press, Cold Spring Harbor, New York, USA. Current advancements in therapeutic antibody design, manufacture, and formulation are described in Sifniotis et al., Antibodies 2019, 8(2), 36; https://doi.org/10.3390/antib8020036, wherein also developments in computational approaches for the strategic design of antibodies with modulated functions are discussed.

The present invention relates to compositions comprising the aforementioned Tau-binding molecule of the present invention, e.g., antibody or Tau-binding fragment, variant or biotechnological derivative thereof, or the polynucleotide(s), vector(s) or cell of the invention as defined hereinbefore. In one embodiment, the composition of the present invention is a pharmaceutical composition and further comprises a pharmaceutically acceptable carrier.

The present invention also provides the pharmaceutical and diagnostic composition, respectively, in form of a pack or kit comprising one or more containers filled with one or more of the above described ingredients, e.g., anti-Tau antibody, Tau-binding fragment, biotechnological derivative or variant thereof, polynucleotide, vector or cell of the present invention. Associated with such container(s) can be a notice in the form prescribed by a governmental agency regulating the manufacture, use or sale of pharmaceuticals or biological products, which notice reflects approval by the agency of manufacture, use or sale for human administration. In addition, or alternatively the kit comprises reagents and/or instructions for use in appropriate immuno-based diagnostic assays. The composition, e.g. kit of the present invention is of course particularly suitable for the risk assessment, diagnosis, prevention and treatment of a disease or disorder which is accompanied with the presence of Tau, and in particular applicable for the treatment of disorders generally associated with Tau as discussed herein above.

The pharmaceutical compositions of the present invention can be formulated according to methods well known in the art; see for example, Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected by different ways, e.g., by intravenous, intraperitoneal, subcutaneous, intramuscular, intranasal, topical or intradermal administration or spinal or brain delivery. Aerosol formulations such as nasal spray formulations include purified aqueous or other solutions of the active agent with preservative agents and isotonic agents. Such formulations are preferably adjusted to a pH and isotonic state compatible with the nasal mucous membranes.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

Several anti-tau antibodies are currently tested in clinical trials, for example antibodies BIIB076 and BIIB092 by Biogen, ABBV-8E12 by AbbVie and R07105705 by Roche/Genentech. Those antibodies were administered by intravenous infusion or subcutaneous injection at different doses. For example, volunteers received single intravenous doses of antibody RO 7105705 ranging from 225 mg to 16800 mg or multiple once-weekly 8400 mg doses. Furthermore, 1200 mg of this antibody were also administered subcutaneously. All doses have been well tolerated and it could be further shown that 13 weeks of treatment with either 3, 10, or 30 mg/kg of the antibody dose-dependently reduced brain pathology. With regard to antibody ABBV-8E12, study participants received a single intravenous dose of either 2.5, 7.5, 15, 25, or 50 mg/kg and antibody BIIB076 was tested at a single dose of 100 mg/kg. Furthermore, participants received doses of 150 mg, 700 mg, or 2100 mg of antibody BIIB092 infused once every four weeks for 12 weeks and there was a marked reduction in CSF free N-terminal fragments of tau, which are proposed to be involved in the spread of pathology in tauopathies, which exceeded 90 percent for all doses.

Thus, in one embodiment, the pharmaceutical composition of the present invention is administered by intravenous infusion or subcutaneous injection, preferably by intravenous infusion either at a single dose of 100 mg, 150 mg, 225 mg, 675 mg, 700 mg, 1200 mg, 2100 mg, 2100 mg, 4200 mg, 8400 mg, or 16800 mg or of 2.0 mg/kg, 2.5 mg/kg, 3 mg/kg, 7.5 mg/kg, 10 mg/kg, 15 mg/kg, 25 mg/kg, 30 mg/kg, 50 mg/kg, 100 mg/kg, 150 mg/kg, 200 mg/kg, or 250 mg/kg or once every four weeks or any dose in between. Alternatively, the pharmaceutical composition is administered at multiple doses in the ranges at mentioned above, preferably at a dose of 8400 mg once weekly, preferably via intravenous infusion. In another embodiment, the pharmaceutical composition is administered subcutaneously at doses in the ranges mentioned above, preferably at a single dose of 1200 mg or once every four weeks.

Neurodegenerative tauopathies are a diverse group of neurodegenerative disorders that share a common pathologic lesion consisting of intracellular aggregates of abnormal filaments that are mainly composed of pathologically hyperphosphorylated Tau in neurons and/or glial cells. Clinical features of the tauopathies are heterogeneous and characterized by dementia and/or motor syndromes. The progressive accumulation of filamentous Tau inclusions may cause neuronal and glial degeneration in combination with other deposits as, e.g., beta-amyloid in AD or as a sole pathogenic entity as illustrated by mutations in the tau gene that are associated with familial forms of FTDP-17. Because of the heterogeneity of their clinical manifestations a potentially non-exhaustive list of tauopathic diseases may be provided including AD, ALS-PDC, AGD, British type amyloid angiopathy, cerebral amyloid angiopathy, DBD, CID, dementia pugilistica, diffuse neurofibrillary tangles with calcification, Down's syndrome, FTD, FTDP-17, frontotemporal lobar degeneration, Gerstmann-Sträussler-Scheinker disease, Hallervorden-Spatz disease, inclusion body myositis, multiple system atrophy, myotonic dystrophy, NP-C, non-Guamanian motor neuron disease with neurofibrillary tangles, PiD, postencephalitic parkinsonism, prion protein cerebral amyloid angiopathy, progressive subcoitical gliosis, PSP, subacute sclerosing panencephalitis, tangle only dementia, multi-infarct dementia and ischemic stroke; see for a review, e.g., Lee et al. (2001) Annu. Rev. Neurosci. 24, 1121-1159 in which Table 1 catalogs the unique members of tauopathies or Sergeant et al. (2005), Bioch. Biophy. Acta 1739, 179-97, with a list in FIG. 2 therein.

The antibody of the present invention may reduce or eliminate at least one symptom of a neurodegenerative tauopathy in a subject. The symptom may be the formation of pathological Tau deposits, hyperphosphorylated Tau deposits, insoluble Tau deposits, neurofibrillary fibers, neurofibrillary fibers, pre-tangle phosphor Tau aggregates, intraneuronal neurofibrillary tangles or extraneuronal neurofibrillary tangles in the brain or spinal cord of a subject; see, e.g., Augustinack et al. (2002) Acta Neuropathol 103, 26-35.

Hence, the present invention also relates to a method of treating a disease or disorder associated with Tau including those recited above, which method comprises administering to a subject in need thereof a therapeutically effective amount of any one of the afore-described Tau-binding molecules, in particular human-derived antibodies of the instant invention. The present invention provides a method of treating a neurodegenerative tauopathy in a subject by administering a therapeutically effective amount of any one of the anti-tau binding molecules of the invention, wherein the administration of the anti-tau antibody ameliorates, treats or reduces the progression of at least one symptom of the neurodegenerative tauopathy in the subject. In principle, the anti-Tau antibody of the present invention is suitable for the treatment of the same diseases and disorders disclosed in the references relating to prior anti-Tau antibodies which are cited herein in section “Background of the invention”, supra.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

### Example 1: Isolation and Identification of Anti-Tau Protein Antibodies

Human-derived antibodies targeting Tau were identified utilizing the Reverse Translational Medicine™ (RTM™) technology, a proprietary technology platform by Neurimmune AG originally described in the international application WO 2008/081008 but modified, further refined and specifically adapted to the target Tau.

### Example 2: Determination of Antibody Sequence and Recombinant Expression

The amino acid sequences of the variable regions of the above-identified anti-Tau antibodies were determined on the basis of their mRNA and cDNA sequences, respectively, obtained from human memory B cells; see FIGS. 1A, B and C. Recombinant expression of complete human IgG1 antibodies with a human or mouse constant domain was performed substantially as described in the Examples of WO 2008/081008, e.g., as described in the Methods section at page 99 and 100.

The framework and complementarity determining regions were determined by comparison with reference antibody sequences available in databases such as Abysis (http://www.bioinf.org.uk/abysis/) and annotated using the Kabat numbering scheme (http://www.bioinf.org.uk/abs/).

### Example 3: Binding Characteristics

To determine the binding specificity and the half maximal effective concentration (EC⁵⁰) of recombinant human-derived Tau antibodies NI-502.4P3, NI-502.31B6, and NI-502.8H1 for Tau or phosphorylated Tau peptides an ELISA EC₅₀ analysis was performed.

In brief, recombinant full-length human Tau (Tau-441, (2N4R) was purchased at rPeptide (Watkinsville, USA) and synthetic phosphorylated Tau peptides were synthesized and purified by Schafer-N(Copenhagen, Denmark): Tau pS396/pS404: GAEIVYK(pS)PVVSGDT(pS)PRHLSNV (SEQ ID NO: 35); Tau pS202/pT205: SGYSSPG(pS)PG(pT)PGSRSRT (SEQ ID NO: 33); Tau pT212/pS214: GTPGSRSR(pT)P(pS)LPTPPTR (SEQ ID NO: 34); Tau pT231: REPKKVAVVR(pT)PPKSPSS (SEQ ID NO: 36); Tau pS422: SSTGSIDMVD(pS)PQLATLA (SEQ ID NO: 37). Tau peptides were then conjugated via a bifunctional linker (SMCC) to bovine serum albumin (BSA).

Indirect ELISA was performed using 96-well half-area microplates (Corning Incorporated, Corning, USA) coated with either recombinant full-length human Tau (rPeptide, Watkinsville, USA) or with BSA (Sigma-Aldrich, Buchs, Switzerland) at a concentration of 3 μg/ml in coating buffer (15 mM Na₂CO₃, 35 mM NaHCO₃, pH 9.42) overnight at 4° C. or with 96-well half-area microplates (Corning Incorporated, Corning, USA) coated with the synthetic BSA-coupled phosphorylated Tau peptides (Schafer-N, Copenhagen, Denmark) or with BSA (Sigma-Aldrich, Buchs, Switzerland) at a concentration of 5 μg/ml in coating buffer (15 mM Na₂CO₃, 35 mM NaHCO₃, pH 9.42) overnight at 4° C. Non-specific binding sites were blocked for 1 h at room temperature with PBS/0.1% Tween®-20 containing 2% BSA (Sigma-Aldrich, Buchs, Switzerland). NI-502.4P3, NI-502.31B6 and NI-502.8H1 antibodies were diluted to the indicated concentrations and incubated for 1 h at room temperature, followed by incubation with a donkey anti-human IgG Fcγ-specific antibody conjugated with HRP (Jackson ImmunoResearch Laboratories, Inc., West Grove, USA). Binding was determined by measurement of HRP activity in a standard colorimetric assay. EC₅₀ values were estimated by non-linear regression using GraphPad Prism software (San Diego, USA).

The binding specificity and EC₅₀ of human-derived, Tau-specific antibodies were determined by indirect ELISA. Antibody NI-502.4P3 (FIG. 2A) specifically recognized the Tau protein with an EC₅₀ of 15.0 nM. Antibody NI-502.31B6 specifically targeted the synthetic phosphorylated peptide Tau pS202/pT205 with an EC₅₀ of 2.0 nM (FIG. 2B) whereas antibody NI-502.8H1 specifically bound the synthetic phosphorylated peptide Tau pT212/pS214 with an EC₅₀ of 2.2 nM (FIG. 2C).

In conclusion, high-throughput immune repertoire analyses of healthy elderly human donor populations by RTM™ screening lead to the successful cloning and recombinant production of human monoclonal antibodies targeting Tau with high affinity. The recombinant human antibodies are either selective for Tau protein or posttranslational modified derivatives.

### Example 4: Assessment of the Binding Epitope of Antibody NI-502.4P3

To determine the binding epitope(s) within the Tau protein that is recognized by the human-derived Tau specific NI-502.4P3 antibody, a pepscan membrane with 108 linear 15 meric peptides with 11 aa overlap between peptides covering the entire Tau protein sequence was used.

In brief, scan of overlapping peptides was used for NI-502.4P3 epitope mapping. The entire sequence of human Tau was synthesized as a total of 108 linear 15 meric peptides with 11 aa overlap between individual peptides (Pepspot™, JPT Peptide Technologies, Berlin, Germany) and spotted onto nitrocellulose membranes. The membrane was activated for 5 min in methanol and then washed at RT in TBS for 10 min. Non-specific binding sites were blocked for 2 hours at RT with Roti®-Block (Carl Roth GmbH+Co. KG, Karlsruhe, Germany) in PBS/0.05% Tween®-20. Human NI-502.4P3 antibody (10 nM) was incubated overnight at 4° C. in Roti®-Block. Binding of NI-502.4P3 was determined using a donkey anti-human IgG Fcγ-specific secondary antibody conjugated with HRP (1:20000 dilution, Jackson ImmunoResearch Laboratories, Inc., West Grove, USA). Blot was developed using ECL and ImageQuant 350 detection (GE Healthcare, Otelfingen, Switzerland)

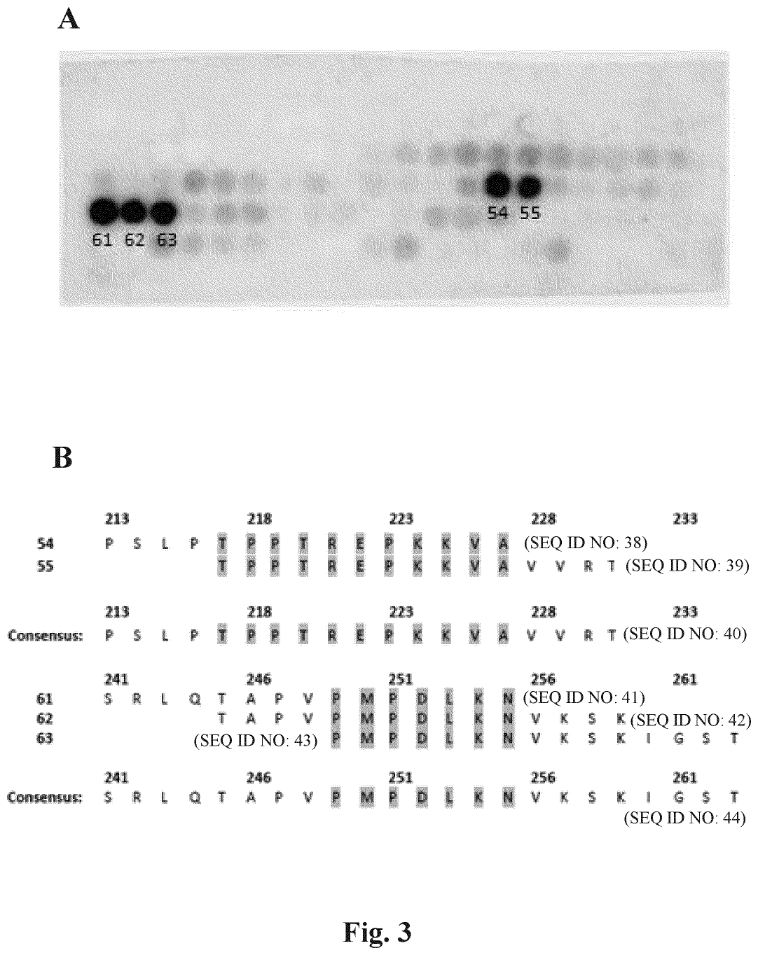

The binding epitope(s) of the human-derived Tau-specific NI-502.4P3 antibody was mapped by the use of a pepscan membrane. Antibody NI-502.4P3 specifically recognized two linear binding epitopes within the human Tau protein sequence: (FIG. 3): 217-TPPTREPKKVA-227 (SEQ ID NO: 31) and 249-PMPDLKN-255 (SEQ ID NO: 32).

### Example 5: Binding Analysis to Tau Pathology in Post Mortem Human Alzheimer's Disease, Progressive Supranuclear Palsy, Pick's Disease and Non-Neurological Control Brain Tissues

To assess the binding of antibodies NI-502.4P3, NI-502.31B6 and NI-502.8H1 to Tau pathology in post-mortem brain tissues derived from selected human AD, PSP and PiD patients and non-neurological controls immunohistochemical analyses were performed.

In brief, formalin fixed, paraffin-embedded 5 μm sections of brain tissues from AD patients (medial temporalis gyms), PiD patients (temporal cortex), PSP patients (temporal cortex) and non-neurological control subjects (amygdala/temporal cortex) (The Netherlands Brain Bank, Amsterdam, The Netherlands) were pretreated for antigen retrieval by cooking in citrate buffer and microwave irradiation for 12 min (600 W). Quenching of endogenous peroxidase activity was achieved by treatment with 3% H₂O₂ in methanol for 10 min at RT. Non-specific binding sites were blocked for 1 h at RT with PBS/5% serum (horse/goat)/4% BSA. After the blocking step, sections were incubated with human-derived NI-502.4P3, NI-502.31B6 and NI-308.8H1 antibodies at 25 nM concentration overnight at 4° C. Detection was performed with biotinylated donkey anti-human IgG (H+L) (1:350 dil, Jackson ImmunoResearch Laboratories, Inc., West Grove, USA) and antibody signal was amplified with the Vectastain Elite ABC kit (Vector Laboratories, Burlingame, USA) and detected with diaminobenzidine (DAB, Thermo Scientific, Rockford, USA). Slides were mounted using Eukitt® mounting medium (O. Kindler GmbH; Freiburg, Germany). Bright-field imaging was performed using a Dotslide VS120 slide scanner (Olympus Schweiz AG, Switzerland).

Neurofibrillary tangles (NFT) composed of hyperphosphorylated Tau filaments are neuropathological hallmarks of AD and other Tauopathies, such as PiD and PSP. Hyperphosphorylated Tau filaments are also the major components of dystrophic neurites and neuropil threads, both of which are common neuropathological features in AD, PiD and PSP. Binding of NI-502.4P3, NI-502.31B6 and NI-502.8H1 to pathological hyperphosphorylated Tau filaments was assessed by immunohistochemical analyses of brain tissue sections from selected patients with AD, PiD and PSP and non-neurological control subjects. As shown in FIGS. 4, 5 and 6, human-derived NI-502.4P3, NI-502.31B6 and NI-502.8H1 antibodies revealed prominent staining of dystrophic neurites, neurofibrillary tangles and neuropil threads in human AD, PSP and Pick's Disease brain tissues. In contrast, non-neurological control brain tissues were negative for the three antibodies tested.

Human-derived antibodies NI-502.4P3, NI-502.31B6 and NI-502.8H1 specifically detected dystrophic neurites, neurofibrillary tangles and neuropil threads in selected human AD, PSP and PiD brain tissues while no staining is observed in non-neurological control subject brain tissues demonstrating the high target specificity of the antibodies for pathologically aggregated and hyperphosphorylated Tau filaments.

### Example 6: In Vitro Target Engagement

Target binding in solution for antibodies NI-502.4P3, NI-502.31B6 and NI-502.8H1 was determined by immunoprecipitation assays in brain tissues of a patient with neuropathologically confirmed AD. Antibodies NI-502.4P3, NI-502.31B6 and NI-502.8H1 specifically capture both Tau and AD-associated Tau in human non-neurological control's and AD patient's brain extracts, respectively.

To determine target binding in solution for antibodies NI-502.4P3, NI-502.31B6 and NI-502.8H1 in brain tissues of a patient with neuropathologically confirmed AD and of a non-neurological control subject immunoprecipitation assays were performed.

In brief, 10% (w/v) brain tissue homogenates of a patient with neuropathologically confirmed AD and of a non-neurological control subject (National Disease Research Interchange, Philadelphia, USA) in PBS buffer (PBS (Gibco®, Life Technologies Europe B.V., Zug, Switzerland) containing Complete protease inhibitor (Roche, Basel, Switzerland) and PhosStop (Roche, Basel, Switzerland)) were prepared. Protein content in the brain tissue homogenates was determined by BCA assay (ThermoFisher Scientific, Waltham, USA). 450 μl aliquots (1.5 mg/ml brain homogenates) were preabsorbed with 50 μl Protein A-Dynabeads (Invitrogen, ThermoFisher Scientific, Waltham, US) by incubation at 4° C. for 2 hours on a rotating platform. To the preabsorbed samples, 10 μg/ml of the selected antibody were added, and samples were incubated overnight at 4° C. on a rotating platform. Immune-complexes were then trap by addition of 50 μl Protein A-Dynabeads by incubation at 4° C. for 1 hour on a rotating platform. Afterwards, beads were washed according to manufacturer's protocol before elution of the immune-complexes.

Immune-complexes were resolved by SDS-PAGE (Novex® Bis-Tris NuPAGE® 4-12%; Invitrogen, ThermoFisher Scientific, Waltham, US) using Novex® NuPAGE® MOPS SDS Running Buffer (Invitrogen, ThermoFisher Scientific, Waltham, US) under non-reducing conditions. Resolved proteins were then electroblotted (iBlot 2 Dry Blotting System, Invitrogen, ThermoFisher Scientific, Waltham, US, 7 min, 20V) on methanol-activated PVDF membranes (Immobilon®-P Transfer Membrane, Merck & Cie, Schaffhausen, Switzerland). Non-specific binding sites were blocked overnight at 4° C. with PBS/0.1% Tween®-20 containing 2% BSA (Sigma-Aldrich, Buchs, Switzerland) (PBST). Tau12 detection antibody (1:5000 dilution, CiteAb, Bath, UK) was incubated for 1 h at RT. Membranes were washed three times in PBST for 15 min at RT and then incubated with a goat anti mouse IgG (H+L) antibody conjugated with HR (1:10,000, Jackson ImmunoResearch Laboratories, Inc., West Grove, USA) for 45 min at RT. Antibody binding was determined by membrane development using ECL and ImageQuant 350 detection (GE Healthcare, Otelfingen, Switzerland).

### Example 7: Antibodies NI-502.4P3, NI-502.31B6 and NI-502.8111 Deplete Seeding-Competent Tau from AD Homogenates

### Brain Homogenate Preparation

Alzheimer's Disease brain tissue (NBB 194-037), inferior frontal gyms) was procured from the Netherlands Brain Bank (NBB). Tissue was weighed and homogenized in 3× mass/volume of PBS containing protease (cOmplete Tablets, Mini EDTA-free, Roche, Switzerland) and phosphatase (PhosSTOP Tablets, Roche, Switzerland) inhibitors. Tissue was homogenized using FastPrep-24 Homogenizer (Lucerna Chem AG) twice with 6.0 m/s for 40 seconds. After homogenization, homogenates were cleared by centrifugation (Microcentrifuge 5430 R (Vaudaux-Eppendorf AG, Switzerland), full speed for 1.5 hours, 4° C.). Protein concentration in the cleared brain homogenate was determined by BCA protein assay (Pierce™ BCA Protein Assay Kit, Thermo Fischer Scientific, USA) according to the manufacturer's instructions. Total Tau concentration was determined using INNOTEST hTAU Ag ELISA (Fujirebio Europe N.V., Belgium) according to the manufacturer's instructions.

### Immunodepletion Studies

Brain homogenates containing 10 ng of tau were mixed with 2-fold serially diluted NI-502.4P3, NI-502.31B6 and NI-502.8H1 (final concentrations of 0.31-80 μg/mL) in 150 μL of Opti-MEM (Invitrogen, Thermo Fisher Scientific, USA) containing protease inhibitors (cOmplete Tablets, Mini EDTA-free, Roche, Switzerland) and allowed to incubate overnight at 4° C. The next day, 50 μL of protein A magnetic bead slurry (Dynabeads™ ProteinA Immunoprecipitation Kit, Thermo Fisher Scientific, USA) was added to each sample to isolate immune complexes. Immunodepleted supernatants were transferred to clean low binding tubes (Vaudaux-Eppendorf AG, Switzerland). Each immunodepletion reaction was performed in duplicate.

### Tau Seeding Assay in HEK293T Biosensor Cells

The HEK293T tau biosensor cell line (HEK293T tau RD-CFP/YFP, ATCC® CRL-3275™) was previously described (Holmes et al., Proc. Natl. Acad. Sci. USA 111 (2014), E4376-85, doi: 10.1073/pnas.1411649111). The cells stably express the repeat domains (RD) of tau protein with a P301S mutation fused to either CFP or YFP. Although at baseline the tau reporter proteins exist in a stable, soluble form within the cell, exposure to exogenous tau seeds leads to tau reporter protein aggregation, which generates a fluorescence resonance energy transfer (FRET signal). Tau aggregation was measured by CFP to YFP FRET signal, detected with fluorescence-activated cell sorting (FACS).

HEK293T tau biosensor cells were plated in 24-well plates (TPP, Switzerland) at 50′000 cells per well in complete HEK Cell culture medium (DMEM/10% FBS/PenStrep/L-Glutamine, Gibco, Thermo Fisher Scientific, USA) and incubated at 37° C., 5% CO₂ for 24 or 48 hours. Immunodepleted brain homogenates (200 μL) were mixed with 6 uL Lipofectamine 2000 (Invitrogen, Thermo Fisher Scientific, USA), gently mixed, incubated for 20 min at RT and then added to the cell media. Cells were cultured for another 24 hours, trypsinized, washed, and subjected to FRET analysis of tau aggregation by FACS. Aggregation was evaluated using a CFP-YFP FRET pair. Signals were measured on an LSR II Fortessa 4 L flow cytometer (BD Biosciences, Switzerland). Forward scatter signal generated by a 488-nm laser line was used as trigger signal. CFP was excited at 405 nm and fluorescence detected with a 450/50-nm bandpass filter indicated no aggregation. YFP excited by CFP emission (FRET), detected with a 525/50-nm bandpass filter, indicated aggregation. The YFP signal was detected using a 530/30-nm bandpass filter. FCS 3.0 files were analyzed using Flowing Software version 2.5.1 (Turku Centre for Biotechnology). Data were reported as integrated FRET density, which was calculated as previously described (Holmes et al., (2014), supra): Integrated FRET density=number of FRET-positive cells×mean FRET signal intensity.

As shown in FIGS. 8A to C, subjecting human brain homogenates derived from Alzheimer's Disease brain tissue with increasing concentrations of antibodies NI-502.4P3, NI-502.31B6 and NI-502.8H1 resulted in a concentration-dependent reduction of Tau aggregation in Tau biosensor cells, which is reflected by the reduction in the integrated FRET density.

1. A human-derived recombinant monoclonal anti-tau antibody or antigen-binding fragment thereof, which is capable of
(i) binding pathological hyperphosphorylated Tau filaments in dystrophic neurites, neurofibrillary tangles and neuropil threads in an immunohistochemical (IHC) assay with brain tissue of patients with Alzheimer's Disease (AD), Progressive supranuclear palsy (PSP) and/or Pick's Disease (PiD); and
(ii) capturing Tau and AD-associated Tau in an immunoprecipitation (IP) assay with brain extracts of patients with AD, and
wherein the antibody or antigen-binding fragment thereof recognizes an epitope comprising the amino acid sequence 217-TPPTREPKKVA-227 (SEQ ID NO: 31) and 249-PMPDLKN-255 (SEQ ID NO: 32) and comprises a variable heavy (VH) chain comprising VH complementarity determining regions (CDRs) 1, 2, and 3, and a variable light (VL) chain comprising VL CDRs 1, 2, and 3, wherein
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions;
wherein the antibody or antigen-binding fragment thereof recognizes an epitope of phosphorylated Tau peptide pS202/pT205 having the amino acid sequence SGYSSPG(pS)PG(pT)PGSRSRT (SEQ ID NO: 33) and comprises a VH chain comprising VH CDRs 1, 2, and 3, and a VL chain comprising VL CDRs 1, 2, and 3, wherein
(g) VH-CDR1 comprises the amino acid sequence of SEQ ID NO: 13 or a variant thereof, wherein the variant comprises one or two amino acid substitutions,
(h) VH-CDR2 comprises the amino acid sequence of SEQ ID NO: 14 or a variant thereof, wherein the variant comprises one or two amino acid substitutions,
(i) VH-CDR3 comprises the amino acid sequence of SEQ ID NO: 15 or a variant thereof, wherein the variant comprises one or two amino acid substitutions,
(j) VL-CDR1 comprises the amino acid sequence of SEQ ID NO: 18 or a variant thereof, wherein the variant comprises one or two amino acid substitutions,
(k) VL-CDR2 comprises the amino acid sequence of SEQ ID NO: 19 or a variant thereof, wherein the variant comprises one or two amino acid substitutions, and
(l) VL-CDR3 comprises the amino acid sequence of SEQ ID NO: 20 or a variant thereof, wherein the variant comprises one or two amino acid substitutions; or
wherein the antibody or antigen-binding fragment thereof recognizes an epitope of phosphorylated Tau peptide pT212/pS214 having the amino acid sequence GTPGSRSR(pT)P(pS)LPTPPTR (SEQ ID NO: 34) and comprises a VH chain comprising VH CDRs 1, 2, and 3, and a VL chain comprising VL CDRs 1, 2, and 3, wherein
(m) VH-CDR1 comprises the amino acid sequence of SEQ ID NO: 23 or a variant thereof, wherein the variant comprises one or two amino acid substitutions,
(n) VH-CDR2 comprises the amino acid sequence of SEQ ID NO: 24 or a variant thereof, wherein the variant comprises one or two amino acid substitutions,
(o) VH-CDR3 comprises the amino acid sequence of SEQ ID NO: 25 or a variant thereof, wherein the variant comprises one or two amino acid substitutions,
(p) VL-CDR1 comprises the amino acid sequence of SEQ ID NO: 28 or a variant thereof, wherein the variant comprises one or two amino acid substitutions,
(q) VL-CDR2 comprises the amino acid sequence of SEQ ID NO: 29 or a variant thereof, wherein the variant comprises one or two amino acid substitutions, and
(r) VL-CDR3 comprises the amino acid sequence of SEQ ID NO: 30 or a variant thereof, wherein the variant comprises one or two amino acid substitutions.

2. A human-derived recombinant monoclonal anti-tau antibody or antigen-binding fragment thereof, wherein the antibody or antigen-binding fragment thereof comprises a variable heavy (VH) chain comprising VH complementarity determining regions (CDRs) 1, 2, and 3, and a variable light (VL) chain comprising VL CDRs 1, 2, and 3, wherein
(a) VH-CDR1 comprises the amino acid sequence of SEQ ID NO: 3,
(b) VH-CDR2 comprises the amino acid sequence of SEQ ID NO: 4,
(c) VH-CDR3 comprises the amino acid sequence of SEQ ID NO: 5,
(d) VL-CDR1 comprises the amino acid sequence of SEQ ID NO: 8,
(e) VL-CDR2 comprises the amino acid sequence of SEQ ID NO: 9, and
(f) VL-CDR3 comprises the amino acid sequence of SEQ ID NO: 10;
wherein the antibody or antigen-binding fragment thereof comprises a VH chain comprising VH CDRs 1, 2, and 3, and a VL chain comprising VL CDRs 1, 2, and 3, wherein
(g) VH-CDR1 comprises the amino acid sequence of SEQ ID NO: 13,
(h) VH-CDR2 comprises the amino acid sequence of SEQ ID NO: 14,
(i) VH-CDR3 comprises the amino acid sequence of SEQ ID NO: 15,
(j) VL-CDR1 comprises the amino acid sequence of SEQ ID NO: 18,
(k) VL-CDR2 comprises the amino acid sequence of SEQ ID NO: 19, and
(l) VL-CDR3 comprises the amino acid sequence of SEQ ID NO: 20; or
wherein the antibody or antigen-binding fragment thereof comprises a VH chain comprising VH CDRs 1, 2, and 3, and a VL chain comprising VL CDRs 1, 2, and 3, wherein
(m) VH-CDR1 comprises the amino acid sequence of SEQ ID NO: 23,
(n) VH-CDR2 comprises the amino acid sequence of SEQ ID NO: 24,
(o) VH-CDR3 comprises the amino acid sequence of SEQ ID NO: 25,
(p) VL-CDR1 comprises the amino acid sequence of SEQ ID NO: 28,
(q) VL-CDR2 comprises the amino acid sequence of SEQ ID NO: 29, and
(r) VL-CDR3 comprises the amino acid sequence of SEQ ID NO: 30.

3. The antibody or antigen-binding fragment thereof of claim 1 or 2, wherein
(i) the VH comprises the amino acid sequence depicted in SEQ ID NO: 2 or a variant thereof, wherein the variant comprises one or more amino acid substitutions; and
(ii) the VL comprises the amino acid sequence depicted in SEQ ID NO: 7, or a variant thereof, wherein the variant comprises one or more amino acid substitutions;
preferably wherein the VH and VL chain amino acid sequence is at least 90% identical to SEQ ID NO: 2 and 7, respectively;
(iii) the VH chain comprises the amino acid sequence depicted in SEQ ID NO: 12 or a variant thereof, wherein the variant comprises one or more amino acid substitutions; and
(iv) the VL comprises the amino acid sequence depicted in SEQ ID NO: 17, or a variant thereof, wherein the variant comprises one or more amino acid substitutions;
preferably wherein the VH and VL chain amino acid sequence is at least 90% identical to SEQ ID NO: 12 and 17, respectively; or
(v) the VH comprises the amino acid sequence depicted in SEQ ID NO: 22 or a variant thereof, wherein the variant comprises one or more amino acid substitutions; and
(vi) the VL comprises the amino acid sequence depicted in SEQ ID NO: 27, or a variant thereof, wherein the variant comprises one or more amino acid substitutions;
preferably wherein the VH and VL chain amino acid sequence is at least 90% identical to SEQ ID NO: 22 and 27, respectively.

4. The antibody or antigen-binding fragment thereof of any one of claims 1 to 3 having an EC₅₀ of about 15 nM for Tau or of about 2 nM for the synthetic phosphorylated peptide Tau pS202/pT205 or Tau pT212/pS214.

5. The antibody or antigen-binding fragment thereof of any one of claims 1 to 4 further comprising an immunoglobulin heavy chain constant region, an immunoglobulin light chain constant region, preferably wherein the immunoglobulin heavy and/or light chain constant region is of the IgG type.

6. The antibody or antigen-binding fragment thereof of any one of claims 1 to 5, which is selected from the group consisting of a single chain Fv fragment (scFv), an F(ab′) fragment, an F(ab) fragment, and an F(ab′)₂ fragment, an Fd, an Fv, a single-chain antibody, and a disulfide-linked Fv (sdFv) and/or which is a chimeric murine-human antibody.

7. One or more polynucleotide(s) encoding the antibody or antigen-binding fragment thereof of any one of claims 1 to 6 or an immunoglobulin VH and VL thereof.

8. One or more polynucleotide(s) of claim 7, wherein the polynucleotide is a cDNA and/or operably linked to a heterologous nucleic acid, preferably expression control sequences.

9. One or more vector(s) comprising the polynucleotide(s) of claim 7 or 8.

10. A host cell comprising the polynucleotide(s) of claim 7 or 8 or the vector(s) of claim 9.

11. A method for preparing an anti-tau antibody, antigen-binding fragment or immunoglobulin chain(s) thereof, said method comprising
(a) culturing the cell of claim 10; and
(b) isolating the antibody, antigen-binding fragment or immunoglobulin chain(s) thereof from the culture.

12. An antibody, antigen-binding fragment or immunoglobulin chain(s) thereof encoded by the polynucleotide(s) of claim 7 or 8 or obtainable by the method of claim 11.

13. An antibody or antigen-binding fragment thereof of any one of claims 1 to 6 or the antibody or antigen-binding fragment thereof of claim 12, which
(i) is detectably labeled with a label selected from the group consisting of an enzyme, a radioisotope, a fluorescent compound, a chemiluminescent compound, a bioluminescent compound, a tag, a flag and a heavy metal;
(ii) is attached to a drug;
(iii) comprises polyethylene glycol;
(iv) comprises a brain targeting entity; and/or
(v) is contained in or conjugated to a vehicle such as an exosome or nanoparticle for delivery to the brain.

14. A composition comprising the antibody or antigen-binding fragment thereof of any one of claim 1 to 6, 12 or 13, the polynucleotide(s) of claim 7 or 8, the vector(s) of claim 9 or the cell of claim 10, preferably the composition is
(i) a pharmaceutical composition and further comprises a pharmaceutically acceptable carrier; or
(ii) a diagnostic composition and designed as a kit, optionally further comprising reagents conventionally used in immuno-based diagnostic methods.

15. An antibody or antigen-binding fragment thereof of any one of claim 1 to 6, 12 or 13, the polynucleotide(s) of claim 7 or 8, the vector(s) of claim 9 or the cell of claim 10 for use in
(i) prophylactic or therapeutic treatment of a neurodegenerative tauopathy in a subject, or
(ii) monitoring the progression of a neurodegenerative tauopathy in a subject, or the response to a treatment of a neurodegenerative tauopathy in a subject,
wherein the neurodegenerative tauopathy is selected from the group consisting of Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Sträussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia and ischemic stroke.
